SEQUENCE LISTING (21 sequences)

SEQ ID NO: 1 - protein, 6 aa, synthetic construct
  REGION 1..6: note = Description of sequence: CDRH1
SDYAWN

SEQ ID NO: 2 - protein, 16 aa, synthetic construct
  REGION 1..16: note = Description of sequence: CDRH2
YISYSGYTSYNPSLKS

SEQ ID NO: 3 - protein, 8 aa, synthetic construct
  REGION 1..8: note = Description of sequence: CDRH3
GGKGPMDY

SEQ ID NO: 4 - protein, 459 aa, synthetic construct
  REGION 1..459: note = Description of sequence: Heavy Chain
MRVLILLWLFTAFPGILSDVQLQESGPGLVKPSQSLSLTCTVTGYSITSDYAWNWIRQFPGNKLEWMGYISYSGYTSYNPSLKSRISITRDTSKNQFFLQLNSVTTEDTTTYYCTRGGKGPMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 5 - protein, 11 aa, synthetic construct
  REGION 1..11: note = Description of sequence: CDRL1
KASQSVSNDVA

SEQ ID NO: 6 - protein, 7 aa, synthetic construct
  REGION 1..7: note = Description of sequence: CDRL2
YASNRYT

SEQ ID NO: 7 - protein, 9 aa, synthetic construct
  REGION 1..9: note = Description of sequence: CDRL3
QQDYSSPYT

SEQ ID NO: 8 - protein, 234 aa, synthetic construct
  REGION 1..234: note = Description of sequence: Light Chain
MKSQTQVFVFLLLCVSGAHGSIVMTQTPKFLLVSAGDRVTITCKASQSVSNDVAWYHQKPGQSPKLLIYYASNRYTGVPDRFTGSGYGTDFTFTISTVQAEDLAVYFCQQDYSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 125 aa, synthetic construct
  REGION 1..125: note = Description of sequence: S100A7 used for study
  SIGNAL 1..18: note = signal peptide
  DOMAIN 120..125: note = Histidine x6 tag
MGWSCIILFLVATATGVHSSNTQAERSIIGMIDMFHKYTRRDDKIEKPSLLTMMKENFPNFLSACDKKGTNYLADVFEKKDKNEDKKIDFSEFLSLLGDIATDYHKQSHGAAPCSGGSQHHHHHH

SEQ ID NO: 10 - DNA, 20 bp, synthetic construct
  misc_feature 1..20: note = Description of sequence: Forward S100A7A ttRF1-S100A7A R1 (WT) primer
  source 1..20: mol_type = other DNA
gagttccaggacagccagag

SEQ ID NO: 11 - DNA, 21 bp, synthetic construct
  misc_feature 1..21: note = Description of sequence: Reverse S100A7A ttRF1-S100A7A R1 (WT) primer
  source 1..21: mol_type = other DNA
ggactccttctattcgccatt

SEQ ID NO: 12 - DNA, 23 bp, synthetic construct
  misc_feature 1..23: note = Description of sequence: Forward S100A7A ttRF1-S100A7A R1 (mutant/floxed) primer
  source 1..23: mol_type = other DNA
gagatggcgcaacgcaattaatg

SEQ ID NO: 13 - DNA, 19 bp, synthetic construct
  misc_feature 1..19: note = Description of sequence: Reverse S100A7A ttRF1-S100A7A R1 (mutant/floxed) primer
  source 1..19: mol_type = other DNA
taggtagcaaagcatgcag

SEQ ID NO: 14 - DNA, 19 bp, synthetic construct
  misc_feature 1..19: note = Description of sequence: Forward CSD-loxF-S100A7A R1 (WT) primer
  source 1..19: mol_type = other DNA
tgatgtagtatggctgcct

SEQ ID NO: 15 - DNA, 20 bp, synthetic construct
  misc_feature 1..20: note = Description of sequence: Reverse CSD-loxF-S100A7A R1 (WT) primer
  source 1..20: mol_type = other DNA
tctcccatctccaacagtcc

SEQ ID NO: 16 - DNA, 25 bp, synthetic construct
  misc_feature 1..25: note = Description of sequence: Forward Sox-2 Cre primer
  source 1..25: mol_type = other DNA
tcatgaactatatccgtaacctgga

SEQ ID NO: 17 - DNA, 25 bp, synthetic construct
  misc_feature 1..25: note = Description of sequence: Reverse Sox-2 Cre primer
  source 1..25: mol_type = other DNA
tgttgccaaactctaaaccaaatac

SEQ ID NO: 18 - DNA, 23 bp, synthetic construct
  misc_feature 1..23: note = Description of sequence: Forward mS100a7a15 prim
  source 1..23: mol_type = other DNA
ggactccctcttccaaatcatac

SEQ ID NO: 19 - DNA, 23 bp, synthetic construct
  misc_feature 1..23: note = Description of sequence: Reverse mS100a7a15 prim
  source 1..23: mol_type = other DNA
cccaagatgtacaggaactcatc

SEQ ID NO: 20 - DNA, 21 bp, synthetic construct
  misc_feature 1..21: note = Description of sequence: Forward 18S rRNA prim
  source 1..21: mol_type = other DNA
cgtcgtagttccgaccataaa

SEQ ID NO: 21 - DNA, 22 bp, synthetic construct
  misc_feature 1..22: note = Description of sequence: Reverse 18S rRNA prime
  source 1..22: mol_type = other DNA
cggaatcgagaaagagctatca

(19) United States
(12) Patent Application Publication
(10) Pub. No.: US 2026/0209330 A1
(43) Pub. Date: Jul. 23, 2026

(54) S100A7 AS A DIAGNOSTIC MARKER AND THERAPEUTIC TARGET FOR DISORDERS

(71) Applicant: Ohio State Innovation Foundation, Columbus, OH (US)

(72) Inventors: Ramesh GANJU, Dublin, OH (US); Sanjay MISHRA, Columbus, OH (US); Manish CHARAN, Columbus, OH (US)

(21) Appl. No.: 19/134,433

(22) PCT Filed: Dec. 1, 2023
(86) PCT No.: PCT/US23/82071

§ 371 Date: May 30, 2025

Related U.S. Application Data
(60) Provisional application No. 63/429,664, filed on Dec. 2, 2022.

(51) Int. Cl. C07K 16/24; A61K 31/7105; A61K 31/713; A61K 38/46; A61K 45/06; A61K 48/00; A61P 3/04; C12N 15/11; G01N 33/68

(52) U.S. Cl. CPC C07K 16/24; A61K 31/7105; A61K 31/713; A61K 38/465; A61K 45/06; A61K 48/005; A61P 3/04; C12N 15/11; G01N 33/6893; C07K 2317/565; C07K 2317/76; G01N 2800/044

(57) ABSTRACT

The present disclosures relates to S100A7 inhibitors and uses thereof for treatment of obesity or obesity-associated diseases. The present disclosures also relates to S100A7 as a biomarker for diagnosing, monitoring, and/or treating obesity or obesity-associated diseases.

### CROSS-REFERENCED TO RELATED APPLICATIONS

This PCT application claims priority to, and the benefit of, U.S. Provisional Patent Application No. 63/429,664, filed Dec. 2, 2022, entitled “S100A7 AS A DIAGNOSTIC MARKER AND THERAPEUTIC TARGET FOR DISORDERS,” which is incorporated by reference herein in its entirety.

### REFERENCE TO SEQUENCE LISTING

The sequence listing submitted on Dec. 1, 2023, as an .XML file entitled “103361-378WO1_ST26.xml” created on Nov. 27, 2023, and having a file size of 29,555 bytes is hereby incorporated by reference pursuant to 37 C.F.R. § 1.52(e)(5).

### FIELD

The present disclosure relates to compositions and methods for treating and/or preventing obesity and/or obesity-associated disorders.

### BACKGROUND

Over 60% of the adult population is considered overweight (body-mass-index (BMI) of 25-29.9) and 38% of the population is considered obese (BMI≥30) in the USA alone and this is especially true in women of African descent who manifest a 58.6% obesity rate. Additionally, breast cancer patients with pre-existing obesity-associated diabetes are at higher risk for distant metastasis and premature mortality. Obesity, while a complex phenotype, is closely associated with inflammation and the availability of several growth factors that support tumor growth. Triple-negative breast cancer (TNBC), which represents approximately 20% of all breast cancer cases, is closely associated with a worse prognosis and low survival due to early metastasis. Epidemiological data have shown a strong association between obesity and TNBC initiation and progression. Moreover, obesity increases 30-50% TNBC associated mortality risks. Few studies also show that TNBC is strongly associated with obesity, metabolic syndrome, and younger age of incidence. Identifying mechanisms and therapeutic targets that link obesity with TNBC and metastasis contributes to developing better therapies for these patients. What is needed are new biomarkers for diagnosing obesity and the associated disorders and compositions for the treatment of the diseases.

### SUMMARY

The present disclosure provides recombinant compositions and methods of use thereof to treat, reduce, decrease, ameliorate, and/or prevent disorders associated with obesity, including but not limited to cancer, autoimmune disorders, and liver diseases.

In one aspect, disclosed herein is a recombinant antibody that specifically binds to a S100A7 polypeptide. In some embodiments, the recombinant antibody comprises at least one heavy chain and/or at least one light chain.

In some embodiments, the heavy chain comprises at least one heavy chain complementarity determining region (CDRH) selected from CDRH1, CDRH2, and CDRH3. In some embodiments, the CDRH1 comprises a sequence at least about 80% identical to SEQ ID NO: 1 or a fragment thereof. In some embodiments, the CDRH2 comprises a sequence at least about 80% identical to SEQ ID NO: 2 or a fragment thereof. In some embodiments, the CDRH3 comprises a sequence at least about 80% identical to SEQ ID NO: 3 or a fragment thereof. In some embodiments, the heavy chain comprises a sequence at least about 80% identical to SEQ ID NO: 4 or a fragment thereof.

In some embodiments, the light chain comprises at least one light chain complementarity determining region (CDRL) selected from CDRL1, CDRL2, and CDRL3. In some embodiments, the CDRL1 comprises a sequence at least about 80% identical to SEQ ID NO: 5 or a fragment thereof. In some embodiments, the CDRL2 comprises a sequence at least about 80% identical to SEQ ID NO: 6 or a fragment thereof. In some embodiments, the CDRL3 comprises a sequence at least about 80% identical to SEQ ID NO: 7 or a fragment thereof. In some embodiments, the light chain comprises a sequence at least about 80% identical to SEQ ID NO: 8 or a fragment thereof.

In one aspect, disclosed herein is a recombinant polynucleotide encoding the recombinant antibody of any preceding aspect.

In one aspect, disclosed herein is a pharmaceutical composition comprising the recombinant antibody of any preceding aspect, or the recombinant polypeptide of any preceding aspect.

In some embodiments, the pharmaceutical composition further comprises a cytosolic phospholipase A2 (cPLA2) inhibitor. In some embodiments, the cPLA2 inhibitor comprises ASB-14780, or a variant thereof. In some embodiments, the pharmaceutical composition further comprises a solute carrier family 6 member 2 (SLC6A2) inhibitor. In some embodiments, the SLC6A2 inhibitor comprises reboxetine, or a variant thereof. In some embodiments, the pharmaceutical composition further comprises an immune checkpoint inhibitor. In some embodiments, the immune checkpoint inhibitor comprises a PD-L1 inhibitor or a PD-1 inhibitor. In some embodiments, the PD-L1 inhibitor comprises Atezolizumab, Avelumab, or Durvalumab. In some embodiments, the PD-1 inhibitor comprises Pembrolizumab, Nivolumab, or Cemiplimab.

In one aspect, disclosed herein is a method of treating obesity or an obesity-associated disorder in a subject in need thereof, comprising administering to the subject a therapeutically effective amount of the pharmaceutical composition of any preceding aspect.

In one aspect, disclosed herein is a method of treating obesity or an obesity-associated disorder in a subject in need thereof comprising determining if the subject has obesity or an obesity-associated disorder, and administering to the subject a therapeutically effective amount of an S100A7 inhibitor if the subject is determined to have obesity or an obesity-associated disorder.

In one aspect, disclosed herein is a method of treating obesity or an obesity-associated disorder in a subject in need thereof comprising obtaining a biological sample from the subject; determining that the subject has obesity or an obesity-associated disorder if there is an increased level of an S100A7 polypeptide in the biological sample in comparison to a reference control; and administering to the subject a therapeutically effective amount of a therapeutic agent to treat obesity or the obesity-associated disorder if the subject is determined to have obesity or an obesity-associated disorder.

In some embodiments, the subject is determined to have obesity or an obesity-associated disorder if the subject has an increased level of an S100A7 polypeptide in a biological sample obtained from the subject in comparison to a reference control. In some embodiments, the therapeutic agent to treat obesity or the obesity-associated disorder is an S100A7 inhibitor. In some embodiments, the S100A7 inhibitor is a polypeptide, a polynucleotide, a small molecule, or a gene editing system. In some embodiments, the polynucleotide comprises a siRNA or a shRNA that targets an S100A7 polynucleotide. In some embodiments, the gene editing system is a CRISPR/Cas endonuclease system. In some embodiments, the CRISPR/Cas endonuclease system comprises a guide RNA targeting an S100A7 polynucleotide. In some embodiments, the polypeptide comprises an antibody that specifically targets S100A7.

In some embodiments, the method comprises an antibody comprising a heavy chain and/or a light chain. In some embodiments, the method comprises the heavy chain comprising at least one heavy chain complementarity determining region (CDRH) selected from CDRH1, CDRH2, and CDRH3. In some embodiments, the CDRH1 comprises a sequence at least about 80% identical to SEQ ID NO: 1 or a fragment thereof. In some embodiments, the CDRH2 comprises a sequence at least about 80% identical to SEQ ID NO: 2 or a fragment thereof. In some embodiments, the CDRH3 comprises a sequence at least about 80% identical to SEQ ID NO: 3 or a fragment thereof. In some embodiments, the heavy chain comprises a sequence at least about 80% identical to SEQ ID NO: 4 or a fragment thereof.

In some embodiments, the method comprises the light chain comprising at least one light chain complementarity determining region (CDRL) selected from CDRL1, CDRL2, and CDRL3. In some embodiments, the CDRL1 comprises a sequence at least about 80% identical to SEQ ID NO: 5 or a fragment thereof. In some embodiments, the CDRL2 comprises a sequence at least about 80% identical to SEQ ID NO: 6 or a fragment thereof. In some embodiments, the CDRL3 comprises a sequence at least about 80% identical to SEQ ID NO: 7 or a fragment thereof. In some embodiments, the light chain comprises a sequence at least about 80% identical to SEQ ID NO: 8 or a fragment thereof.

In some embodiments, the method further comprises administering to the subject a therapeutically effective amount of a cytosolic phospholipase A2 (cPLA2) inhibitor. In some embodiments, the cPLA2 inhibitor comprises ASB-14780. In some embodiments, the method further comprises administering to the subject a therapeutically effective amount of a solute carrier family 6 member 2 (SLC6A2) inhibitor. In some embodiments, the SLC6A2 inhibitor comprises reboxetine. In some embodiments, the method further comprises administering to the subject a therapeutically effective amount of an immune checkpoint inhibitor. In some embodiments, the immune checkpoint inhibitor comprises a PD-L1 inhibitor or a PD-1 inhibitor. In some embodiments, the PD-L1 inhibitor is Atezolizumab, Avelumab, or Durvalumab. In some embodiments, the PD-1 inhibitor is Pembrolizumab, Nivolumab, or Cemiplimab.

In some embodiments, the obesity-associated disorder comprises an autoimmune disease, cancer, or fatty liver disease. In some embodiments, the cancer comprises breast cancer, liver cancer, lung cancer, skin cancer, bladder cancer, stomach cancer, or head and neck cancer. In some embodiments, the subject has psoriasis or diabetes.

### BRIEF DESCRIPTION OF FIGURES

The accompanying drawings, which are incorporated in and constitute a part of this specification, illustrate several embodiments and together with the description illustrate the disclosed compositions and methods.

FIGS. 1A, 1B, 1C, and 1D show that high expression of S100A7 is associated with obesity and increased body weight. Figure iA shows the immunoblot analysis of S100A7 in visceral WAT and mammary glands harvested. FIG. 1B shows the wild type and genetically obese ob/ob mice.

FIG. 1C shows the mice fed with either chow (normal diet) or high-fat diet (HFD). GAPDH or β-actin were used as loading controls. FIGS. 1C and 1D show the expression analysis of S100A7 in fat and skin tissues of female subjects using the publicly available GSE151839 GEO dataset. Relative expression is presented as an arbitrary unit (A.U.) and a non-parametric test (Mann-Whitney U test) was applied to calculate the p values. ns: non-significant, **** P<0.0001.

FIGS. 2A, 2B, 2C, 2D, 2E, and 2F show the generation of mS100a7a15 (S100A7) conditional knock out (KO) mouse model. FIG. 2A shows the targeting strategy. The targeting vector (conditional ready, tm1a) includes a ~5.9 kb 5′ and a ~4.2 kb 3′ homology arms. The mutated locus contains an En2-IRES-LAcZ cassette to survey S100a7a gene expression. The targeting vector also includes a DTa cassette (not depicted) for in vitro negative selection of embryonic stem (ES) clones with random integrations. Exon 3 of the gene S100a7a is flanked by 2 loxP sites (red triangles) and can be removed by Cre recombination in a tissue-specific manner.

FIG. 2B shows the representative southern blot screening of targeted S1B6a ES cells. DNA was digested with NheI (N in A) and hybridized with a 5′ probe. Clone G12 shows the targeted mutated band of 9.5 kb and was used to generate the S100a7a floxed mouse line. The presence of the distal loxP on the 3′ of exon 3 was confirmed by PCR. FIG. 2C shows the qRT-PCR analysis of S100A7 expression in lung tissue of S100A7 floxed (SA7^{F/F}) and KO (SA7^{-/-}) mice after SOX2-Cre mediated whole-body deletion of S100A7 gene. Fold changes were normalized to 18S gene expression. FIG. 2D shows the immunoblot analysis of S100A7 expression in mammary gland samples extracted from S100A7 floxed (f/f) and KO mice. GAPDH was used as a loading control. FIG. 2E shows the immunohistochemistry (IHC) analysis of S100A7 expression in mammary glands harvested from S100A7 floxed (f/f) and KO mice. FIG. 2F shows the histological analysis of H/E-stained mammary glands harvested from S100A7 floxed (f/f) and KO mice. *** is p<0.001. t-test was used to calculate the p-value.

FIGS. 3A, 3B, 3C, 3D, 3E, 3F, 3G, 3H, 3I, and 3J show loss of S100A7 protects mice against HFD-induced obesity. FIGS. 3A, 3B, and 3C show the image and comparison of body weight changes between S100A7 floxed (f/f) and whole-body KO mice fed with HFD or chow. The bar diagram represents the average body weight of each group −14 weeks of chow or HFD consumption. FIG. 3D show the bar diagram shows the fat mass of S100A7^{f/f} and KO mice analyzed by echoMRI. FIGS. 3E, 3F, 3G, 3H, 3I, and 3J show the photomicrographs and bar diagrams showing the changes in weight of visceral WAT (vWAT), mammary glands (MGs), and sub-cutaneous WAT (scWAT) mass (gm) between S100A7^{f/f} and KO mice fed either with chow or HFD. ns: non-significant, *P<0.05, ** P<0.01, *** P<0.001, **** P<0.0001. One-way ANOVA was used for calculating statistical significance.

FIGS. 4A, 4B, 4C, and 4D show the effect of S100A7 ablation on bone growth and HFD intake. FIGS. 4A and 4B show the photomicrographs and changes in lower limb (tibia) Length (cm) between S100A7^{f/f} and KO mice fed either with chow or HFD. FIG. 4C shows the bar diagram shows HFD intake between S100A7^{f/f} and KO mice with HFD for 10 days. FIG. 4D shows the bar diagram shows the ratio of HFD intake vs body weight of S100A7^{f/f} and KO mice fed with HFD for 10 days. ns: non-significant, *P<0.05. One-way ANOVA and t-test were used for calculating statistical significance.

FIGS. 5A, 5B, 5C, 5D, 5E, and 5F show that S100A7 deletion protects mice against HFD-induced obesity-associated fatty liver. FIGS. 5A and 5B show the photomicrographs and changes in liver mass (gm) between S100A7^{f/f} and KO mice fed either with chow or HFD. FIG. 5C shows the oil-red O staining of liver samples harvested from S100A7^{f/f} and KO mice fed either with chow or HFD. FIG. 5D shows the bar diagram shows the length of lipid droplets accumulated in livers of S100A7^{f/f} and KO mice fed either with chow or HFD. FIGS. 5E and 5F show the immunoblot analysis of CD36 and FATP1 in liver tissue extracts of S100A7^{f/f} and KO mice fed either with chow or HFD. GAPDH or 3-actin were used as loading controls. ns: non-significant, *P<0.05, **** P<0.0001. One-way ANOVA was used for calculating statistical significance.

FIGS. 6A, 6B, 6C, and 6D show the effect of S100A7 deletion on HFD-induced expression of adipokines and glucose/insulin tolerance. FIGS. 6A and 6B show that all the mice were fasted overnight, wherein a glucose tolerance test (GTT) and an insulin tolerance test (IIT) was performed on S100A7^{f/f} and KO mice fed either with chow or HFD for 12 weeks. FIG. 6C shows that at the end of the HFD-induced obesity experiment, all the mice were euthanized, and blood plasma samples were analyzed for the expression of different adipokines using a mouse adipokine array. Bar diagram showing the densitometric analysis of adiponectin, RAGE and leptin identified after adipokine array. FIG. 6D shows the qRT-PCR analysis of Leptin gene in vWAT samples harvested from S100A7^{f/f} and KO mice fed either with chow or HFD. Ppia and TBP were used for geometric mean normalization. ns: non-significant, *P<0.05, ** P<0.01. One-way ANOVA was used for calculating statistical significance.

FIGS. 7A, 7B, and 7C show effect of S100A7 deletion on HFD-induced expression of different adipogenic factors and mitochondrial oxidative phosphorylation (OXPHOS) protein complexes. Immunoblot analysis of PPARγ, perilipin-1 and five different OXPHOS complexes in brown adipose tissue (BAT), subcutaneous white adipose tissue (scWAT), and visceral white adipose tissue (vWAT) of S100A7^{f/f} and KO mice fed either with chow or HFD. GAPDH, α-tubulin and β-actin were used as loading controls.

FIGS. 8A, 8B, 8C, 8D, 8E, 8F, and 8G show high expression of S100A7 associated with triple-negative breast cancer growth and monocyte abundance. FIG. 8A shows the mRNA levels of S100A7 in African American (AA; n=123) and Caucasian (CA; n=712) triple-negative breast cancer (TNBC) patients using TCGA dataset. FIG. 8B shows the S100A7 levels were analyzed in plasma samples of AA-TNBC (N=29) and CA-TNBC (N=59) by ELISA. Murine TNBC Mvt-1 cell line was injected into the 4th MGs of S100A7 floxed mice (f/f) and whole-body knock-out (KO) mice. FIG. 8C shows the photomicrographs of breast tumors were taken. FIGS. 8D and 8E show that tumor volume and tumor weight was measured. FIGS. 8F and 8G show the bar diagrams of the number/abundance of peripheral blood monocytes (K/μL; 1000 cells per microliter of blood) and Spleen-derived monocytes (CD11b⁺Gr-1⁺ Ly6C⁺ Ly6G⁻ out of CD45⁺ total leucocytes) were measured in S100A7 floxed mice (f/f) and whole-body knock-out (KO) mice. *P<0.05, ** P<0.01, **** p<0.0001. t-test and non-parametric tests were used for calculating statistical significance.

FIGS. 9A, 9B, 9C, 9D, 9E, 9F, 9G, and 9H show that neutralization of S100A7 decreases the S100A7-induced TNBC growth and metastasis. MDA-MB-231 cells were treated with hS100A7 neutralizing antibody (clone 28F8-2; 300 ng/μl) or control IgG or PBS in the presence or absence of recombinant human S100A7 (100 ng/ml) and spheroid invasion assay was performed by using 3D-Cultrex spheroid invasion kit. FIG. 9A shows the photomicrographs of invaded spheroids. FIG. 9B show the bar diagram representing the invaded area of spheroids. The area between red and blue circles represents the invaded area. FIG. 9C shows the MDA-MB-231 cells were treated with hS100A7 neutralizing antibody (clone 28F8-2; 300 ng/μl) or control IgG or PBS in the presence or absence of recombinant human S100A7 (100 ng/ml) and subjected to migration assay. FIG. 9D shows the MDA-MB-231 cells were treated with hS100A7 nAb (clone 28F8-2; 300 ng/μl) or control IgG or PBS in the presence or absence of recombinant human S100A7 (100 ng/ml) and expression of PD-L1 was determined by using Western blot. GAPDH was used as a loading control. S100A7 overexpressing MDA-MB-231 cells (1×10⁶) were injected into the female NCG mice. After the onset of palpable tumors, mice were either treated with vehicle control (VC), S100A7 nAb (100 g/mouse, i.p. route; clone 28F8-2; two times a week) alone or in combination with ASB-14780 (50 mg/kg·bt, oral route; three times a week). FIG. 9E shows the representative image of tumors harvested from control IgG, S100A7 nAb, ASB-14780, and combination-treated groups. FIGS. 9F, 9G, and 9H show the analysis of tumor volume, tumor weight, and liver metastasis of control IgG, S100A7 nAb, ASB-14780, and combination-treated. *** is p<0.001; **** is p<0.0001. One way ANOVA and t test were used for calculating statistical significance.

FIGS. 10A, 10B, 10C, 10D, 10E, 10F, 10G, and 10H show that neutralization of S100A7 attenuated the H1FD-induced obesity and obesity-associated TNBC growth and metastasis. Six weeks old wild type female FVB mice were provided with HFD for six weeks and then at the end of 6^{th} week, these mice were divided into four groups and were treated with vehicle control (VC), S100A7 nAb (100 g/mouse; i.p. route; clone 28F8-2; two times a week) alone or reboxetine (20 mg/kg·bt, i.p.; twice a week) alone or in combination for up to 20 weeks. FIG. 10A shows the representative image of mice provided with HFD and then treated with VC, S100A7 nAb, Reb and their combo at the end of 20^{th} week. FIG. 10B shows the representative images of visceral white adipose tissue (vWAT) and breast tumors harvested from mice provided with HFD and then treated with VC, S100A7 nAb, Reb and combo for 14 weeks. FIGS. 10C, 10D, 10E, 10F, and 10G show the analysis of body weight, vWAT mass (gm), tumor volume, tumor weight (gm), and Lung metastasis of VC, S100A7 nAb, Reb and combo-treated animals. *P<0.05, ** P<0.01. One way ANOVA was used for calculating statistical significance.

FIGS. 11A, 11B, 11C, 11D, and 11E show the loss of S100A7 protects mice against THFD-induced changes in adipogenic markers and brown adipose tissue (BAT) histology. FIGS. 11A and 11B shows the immunoblot analysis of UCP1 expression in BAT and Subcutaneous white adipose tissue (scWAT) samples extracted from S100A7 floxed (f/f) and KO mice fed with chow or HFD. FIGS. 11C and 11D show the immunoblot analysis of CD36 expression in scWAT and visceral white adipose tissue (vWAT) samples extracted from S100A7 floxed (f/f) and KO mice fed with chow or HFD. GAPDH, α-tubulin and β-actin were used as loading controls. FIG. 11E shows the histological analysis of H&E (100×) images of BAT samples harvested from S100A7 floxed (f/f) and KO mice fed with chow or HFD. Representative photomicrographs of the difference were sobered in BAT from S100A7 f/f and KO animals on normal chow and H1FD. In animals given normal chow, brown and white adipose were fairly well-demarcated, with areas of white adipose (blue arrow) mixing with brown fat at the periphery. An area of brown fat (black arrowhead) was seen within the larger white adipose region as well. In the H1FD-fed animals, the appearance of the brown fat was altered. In the S100A7 f/f mice, diffuse enlargement of lipid droplets in brown adipocytes (black arrow), including extreme enlargement mirroring white adipose lipid droplets (green arrow) was noticed. In the S100A7 KO mice fed with HFD, this enlargement was less severe and pronounced, with areas of more normal brown fat morphology remaining (black arrowhead); markedly enlarged lipid droplets (green arrow) and areas of brown adipose morphology with individually enlarged lipid droplets (black arrow) were present.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are several values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims that follow, reference will be made to a number of terms that shall be defined to have the following meanings:

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and administration by another.

“Optional” or “optionally” means that the subsequently described event or circumstance may or may not occur, and that the description includes instances where said event or circumstance occurs and instances where it does not.

The term “biocompatible” generally refers to a material and any metabolites or degradation products thereof that are generally non-toxic to the recipient and do not cause significant adverse effects to the subject.

The term “biomarker” refers to a biological marker characterizing a phenotype. A biomarker typically includes a gene or a gene product. Depending on the gene, “detecting a biomarker” may include detecting altered gene expression, epigenetic modifications, germ-line or somatic mutations, etc. In the case of a gene product, “detecting a biomarker” may mean detecting the presence, quantity or change in the quantity of a cell surface marker, a soluble compound such as cytokine, etc. “Detecting a biomarker” may also include detecting gene expression (mRNA or protein) or a metabolite reflective of a gene's expression or activity.

The term “cancer” as used herein is defined as a disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, reagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, reagents, conjugates, active metabolites, isomers, fragments, analogs, etc.

A “control” is an alternative subject or sample used in an experiment for comparison purposes.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional properties, such as removing or adding amino acids capable of disulfide bonding, increasing its bio-longevity, altering its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

A “nucleic acid” is a deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered sugar moieties, such as a locked nucleic acid (LNA).

A “nucleotide” is a fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

The term “polynucleotide” refers to a single or double stranded polymer composed of nucleotide monomers (DNA or RNA).

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down-regulate the activity of the described target protein, e.g., antagonists. A control sample (untreated with inhibitors) is assigned a relative activity value of 100%. Inhibition of a described target protein is achieved when the activity value or expression level relative to the control is about 80%, optionally 50% or 25, 10%, 5% or 1%.

“Sequence identity” is defined as the similarity between two nucleic acid sequences is expressed in terms of the similarity between the sequences, otherwise referred to as sequence identity. Sequence identity is frequently measured in terms of percentage identity, similarity, or homology; a higher percentage identity indicates a higher degree of sequence similarity. The NCBI Basic Local Alignment Search Tool (BLAST), Altschul et al, J. Mol. Biol. 215:403-10, 1990, is available from several sources, including the National Center for Biotechnology Information (NCBI, Bethesda, MD), for use in connection with the sequence analysis programs blastp, blastn, blastx, tblastn and tblastx. It can be accessed through the NCBI website. A description of how to determine sequence identity using this program is also available on the website. When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website. These sequence identity ranges are provided for guidance only; it is entirely possible that strongly significant homologs could be obtained that fall outside of the ranges provided.

A “subject” is any mammal, such as humans, non-human primates, pigs, sheep, horses, dogs, cats, cows, rodents and the like.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively, or remedially. Prophylactic treatments are administered to a subject prior to onset, during early onset, or after an established development of a disorder or symptoms thereof. Prophylactic administration can occur for several days to years prior to the manifestation of symptoms of a disorder.

“Therapeutically effective amount” or “therapeutically effective dose” of a composition (e.g., a composition comprising the recombinant antibody disclosed herein) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Antibodies

Disclosed herein relates to a recombinant antibody that specifically binds to an S100A7 polypeptide. In some embodiments, the S100A7 polypeptide comprises an amino acid sequence of about 60% (for example, at least about 60%, about 65%, about 70%, about 75%, about 80%, about 85%, about 90%, about 95%, about 98%, or about 99%) identical to SEQ ID NO: 9 or a fragment thereof. Also disclosed herein is a recombinant polynucleotide encoding the recombinant antibody disclosed herein.

“S100A7” refers herein to a polypeptide that, in humans, is encoded by the S100A7 gene. In some embodiments, the S100A7 polypeptide is that identified in one or more publicly available databases as follows: HGNC: 10497, NCBI Entrez Gene: 6278, Ensembl: ENSG00000143556, OMIM®: 600353, UniProtKB/Swiss-Prot: P31151. In some embodiments, the S100A7 polypeptide comprises the sequence of SEQ ID NO: 9, or a polypeptide sequence having at or greater than about 80%, about 85%, about 90%, about 95%, or about 98% homology with SEQ ID NO: 9, or a polypeptide comprising a portion of SEQ ID NO: 9. The S100A7 polypeptide of SEQ ID NO: 9 may represent an immature or pre-processed form of mature S100A7, and accordingly, included herein are mature or processed portions of the S100A7 polypeptide in SEQ ID NO: 9.

In some embodiments, the recombinant antibody comprises at least one heavy chain and/or at least one light chain. In some embodiments, the heavy chain comprises a heavy chain complementarity determining region 1(CDRH1), a heavy chain complementarity determining region 2(CDRH2), and/or a heavy chain complementarity determining region 3(CDRH3). In some embodiments, the CDRH1 comprises a sequence at least about 80% (for example, at least about 80%, about 85%, about 90%, about 95%, about 98%, or about 99%) identical to SEQ ID NO: 1 or a fragment thereof. In some embodiments, the CDRH2 comprises a sequence at least about 80% (for example, at least about 80%, about 85%, about 90%, about 95%, about 98%, or about 99%) identical to SEQ ID NO: 2 or a fragment thereof. In some embodiments, the CDRH3 comprises a sequence at least about 80% (for example, at least about 80%, about 85%, about 90%, about 95%, about 98%, or about 99%) identical to SEQ ID NO: 3 or a fragment thereof. In some embodiments, the heavy chain comprises a sequence of at least about 80% (for example, at least about 80%, about 85%, about 90%, about 95%, about 98%, or about 99%) identical to SEQ ID NO: 4 or a fragment thereof.

In some embodiments, the light chain comprises alight chain complementarity determining region 1(CDRL1), a light chain complementarity determining region 2(CDRL2), and/or a light chain complementarity determining region 3(CDRL3). In some embodiments, the CDRL1 comprises a sequence of at least about 80% (for example, at least about 80%, about 85%, about 90%, about 95%, about 98%, or about 99%) identical to SEQ ID NO: 5 or a fragment thereof. In some embodiments, the CDRL2 comprises a sequence of at least about 80% (for example, at least about 80%, about 85%, about 90%, about 95%, about 98%, or about 99%) identical to SEQ ID NO: 6 or a fragment thereof. In some embodiments, the CDRL3 comprises a sequence of at least about 80% (for example, at least about 80%, about 85%, about 90%, about 95%, about 98%, or about 99%) identical to SEQ ID NO: 7 or a fragment thereof. In some embodiments, the light chain comprises a sequence at least about 80% (for example, at least about 80%, about 85%, about 90%, about 95%, about 98%, or about 99%) identical to SEQ ID NO: 8 or a fragment thereof.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (1), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG1, IgG2, IgG3, and IgG4; IgA1 and IgA2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, and the like, including hybrid fragments. Thus, fragments of the antibodies that retain the ability to bind their specific antigens are provided. For example, fragments of antibodies that maintain S100A7 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well-known in the art. Generally, a humanized antibody has one or more amino acid residues introduced into it from a non-human source.

These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Disclosed are hybridoma cells that produces the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises an S100A7 polypeptide. Traditionally, the generation of monoclonal antibodies has depended on the availability of purified protein or peptides for use as the immunogen. More recently DNA based immunizations have shown promise as a way to elicit strong immune responses and generate monoclonal antibodies.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for S100A7 or a portion thereof and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonyl) chemistry. (Applied Biosystems, Inc., Foster City, CA). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allows relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with an S100A7 polypeptide. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity is assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

### Pharmaceutical Compositions

In some aspects, disclosed herein is a pharmaceutical composition comprising the recombinant antibody disclosed herein or the recombinant polynucleotide disclosed herein.

In some embodiments, the pharmaceutical composition comprises a cytosolic phospholipase A2 (cPLA2) inhibitor (including, for example, ASB-14780). In some embodiments, the pharmaceutical composition further comprises a solute carrier family 6 member 2 (SLC6A2) inhibitor (including, for example, reboxetine).

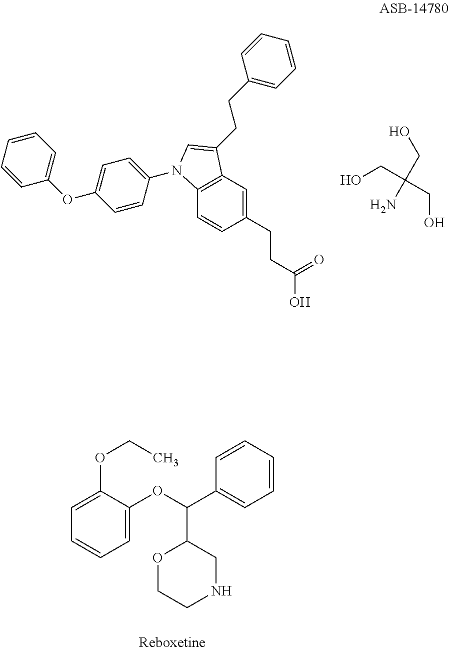 [SMILES: O=C(O)CCc1ccc2c(c1)c(CCc1ccccc1)cn2-c1ccc(Oc2ccccc2)cc1 ; NC(CO)(CO)CO ; CCOc1ccccc1OC(c1ccccc1)C1CNCCO1]

In some embodiments, the pharmaceutical composition further comprises an immune checkpoint inhibitor. In some embodiments, the immune checkpoint inhibitor is a PD-L1 inhibitor or a PD-1 inhibitor. Checkpoint inhibitors include, but are not limited to antibodies that block PD-1 (Pembrolizumab, Nivolumab, or Cemiplimab), PD-L1 (Atezolizumab, Avelumab, or Durvalumab), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

As used herein, the term “PD-1 inhibitor” refers to a composition that binds to PD-1 and reduces or inhibits the interaction between the bound PD-1 and PD-L1. In some embodiments, the PD-1 inhibitor is an anti-PD-1 antibody. In some embodiments, the PD-1 inhibitor is a monoclonal antibody that is specific for PD-1 and that reduces or inhibits the interaction between the bound PD-1 and PD-L1. Non-limiting examples of anti-PD1 antibody are pembrolizumab, nivolumab, and cemiplimab. In some embodiments, the pembrolizumab is KEYTRUDA® or a bioequivalent.

In some embodiments, the pembrolizumab is that described in U.S. Pat. Nos. 8,952,136, 8,354,509, or U.S. Pat. No. 8,900,587, all of which are incorporated by reference in their entireties. In some embodiments, the pembrolizumab has the Unique Ingredient Identifier (UNII) of the U.S. Food and Drug Administration of DPT0O3T46P. In some embodiments, the nivolumab is OPDIVO® or a bioequivalent. In some embodiments, the nivolumab has the Unique Ingredient Identifier (UNII) of the U.S. Food and Drug Administration of 31YO63LBSN. In some embodiments, the nivolumab is that described in U.S. Pat. Nos. 7,595,048, 8,738,474, 9,073,994, 9,067,999, 8,008,449, or U.S. Pat. No. 8,779,105, all of which are incorporated by reference in their entireties. In some embodiments, the cemiplimab is LIBTAYO® or a bioequivalent. In some embodiments, the cemiplimab has the Unique Ingredient Identifier (UNII) of the U.S. Food and Drug Administration of 6QVL057INT. In some embodiments, the cemiplimab is that described in U.S. patent Ser. No. 10/844,137, which is incorporated by reference in its entirety.

The term “PD-L1 inhibitor” refers to a composition that binds to PD-L1 and reduces or inhibits the interaction between the bound PD-L1 and PD-1. In some embodiments, the PD-L1 inhibitor is an anti-PD-L1 antibody. In some embodiments, the anti-PD-L1 antibody is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ® or a bioequivalent. In some embodiments, the atezolizumab has the Unique Ingredient Identifier (UNII) of the U.S. Food and Drug Administration of 52CMI0WC3Y. In some embodiments, the atezolizumab is that described in U.S. Pat. No. 8,217,149, which is incorporated by reference in its entirety. In some embodiments, the avelumab is BAVENCIO® or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI® or a bioequivalent. In some embodiments, the durvalumab has the Unique Ingredient Identifier (UNII) of the U.S. Food and Drug Administration of 28×28×90 KV. In some embodiments, the durvalumab is that described in U.S. Pat. No. 8,779,108, which is incorporated by reference in its entirety.

The term “CTLA-4 inhibitor” refers to a composition that binds to CTLA-4 and reduces or inhibits the interaction between the bound CTLA-4 and CD80/86. In some embodiments, the CTLA-4 inhibitor is an anti-CTLA-4 antibody. In some embodiments, the anti-CTLA-4 antibody is a monoclonal antibody that is specific for CTLA-4 and that reduces or inhibits the interaction between the bound CTLA-4 and CD80/86. In some embodiments, the anti-CTLA-4 antibody is ipilimumab. In some embodiments, the ipilimumab is Yervoy® or a bioequivalent. In some embodiments, the ipilimumab has the Unique Ingredient Identifier (UNII) of the U.S. Food and Drug Administration of 6T8C155666. In some embodiments, the ipilimumab is that described in U.S. Pat. No. 6,984,720, which is incorporated by reference in its entirety.

Methods of Treating and/or Preventing Diseases

Also disclosed herein are methods of treating and/or preventing obesity or obesity-associated diseases (including, for example, cancers, autoimmune diseases, or fatty liver disease) in a subject in need thereof by using the recombinant antibodies, polynucleotides, or pharmaceutical compositions disclosed herein. These methods have been shown to be effective at treating, mitigating, and/or preventing obesity or obesity-associated diseases (including, for example, cancers, autoimmune diseases, or fatty liver disease).

In some aspects, disclosed herein is a method of treating and/or preventing obesity or an obesity-associated disorder in a subject in need thereof comprising determining if the subject has obesity or an obesity-associated disorder; and administering to the subject a therapeutically effective amount of an S100A7 inhibitor if the subject is determined to have obesity or an obesity-associated disorder.

By “treat” or “prevent” is meant that the severity of the disease is reduced or prevented by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, as compared to a control, or to a subject before treatment.

The term “obesity” is defined as abnormal or excessive fat accumulation in a subject. A body mass index (BMI) over 25 is considered overweight, and over 30 is obese. Obesity has been found to be associated with disorders including, for example, high blood pressure (hypertension); high LDL cholesterol, low HDL cholesterol, or high levels of triglycerides (dyslipidemia); type 2 diabetes; coronary heart disease; stroke; gallbladder disease; osteoarthritis; sleep apnea and breathing problems; many types of cancer; and mental illness such as clinical depression, anxiety, and other mental disorders. Accordingly, it should be understood that a treatment using the compositions disclosed herein may be a treatment of one or more of obesity, high blood pressure (hypertension), high LDL cholesterol, low HDL cholesterol, high levels of triglycerides (dyslipidemia), type 2 diabetes, psoriasis, coronary heart disease, stroke, gallbladder disease, osteoarthritis, sleep apnea and breathing problems, cancer (such as TNBC), and mental illness (such as clinical depression, anxiety, or other mental disorders). Treatment of obesity can be indicated by decrease in body weight, reduction in white fat, and/or increase in brown fat.

In some embodiments, the subject is determined to have obesity or an obesity-associated disorder if the subject has an increased level of an S100A7 polypeptide in a biological sample obtained from the subject in comparison to a reference control (e.g., a healthy subject).

In some embodiments, the S100A7 inhibitor is a polypeptide, a polynucleotide, a small molecule, or a gene editing system. In some embodiments, the polynucleotide is a siRNA or a shRNA that targets an S100A7 polynucleotide. In some embodiments, the gene editing system is a CRISPR/Cas endonuclease system. In some embodiments, the CRISPR/Cas endonuclease system comprises a guide RNA targeting an S100A7 polynucleotide (for example, an exon or a UTR of an S100A7 gene).

In some embodiments, the polypeptide is a recombinant antibody that specifically targets S100A7.

In some embodiments, the recombinant antibody comprises at least one heavy chain and/or at least one light chain. In some embodiments, the heavy chain comprises a heavy chain complementarity determining region 1(CDRH1), a heavy chain complementarity determining region 2(CDRH2), and/or a heavy chain complementarity determining region 3(CDRH3). In some embodiments, the CDRH1 comprises a sequence at least about 80% (for example, at least about 80%, about 85%, about 90%, about 95%, about 98%, or about 99%) identical to SEQ ID NO: 1 or a fragment thereof. In some embodiments, the CDRH2 comprises a sequence at least about 80% (for example, at least about 80%, about 85%, about 90%, about 95%, about 98%, or about 99%) identical to SEQ ID NO: 2 or a fragment thereof. In some embodiments, the CDRH3 comprises a sequence at least about 80% (for example, at least about 80%, about 85%, about 90%, about 95%, about 98%, or about 99%) identical to SEQ ID NO: 3 or a fragment thereof. In some embodiments, the heavy chain comprises a sequence of at least about 80% (for example, at least about 80%, about 85%, about 90%, about 95%, about 98%, or about 99%) identical to SEQ ID NO: 4 or a fragment thereof.

In some embodiments, the light chain comprises alight chain complementarity determining region 1(CDRL1), a light chain complementarity determining region 2(CDRL2), and/or a light chain complementarity determining region 3(CDRL3). In some embodiments, the CDRL1 comprises a sequence of at least about 80% (for example, at least about 80%, about 85%, about 90%, about 95%, about 98%, or about 99%) identical to SEQ ID NO: 5 or a fragment thereof. In some embodiments, the CDRL2 comprises a sequence of at least about 80% (for example, at least about 80%, about 85%, about 90%, about 95%, about 98%, or about 99%) identical to SEQ ID NO: 6 or a fragment thereof. In some embodiments, the CDRL3 comprises a sequence of at least about 80% (for example, at least about 80%, about 85%, about 90%, about 95%, about 98%, or about 99%) identical to SEQ ID NO: 7 or a fragment thereof. In some embodiments, the light chain comprises a sequence at least about 80% (for example, at least about 80%, about 85%, about 90%, about 95%, about 98%, or about 99%) identical to SEQ ID NO: 8 or a fragment thereof.

In some examples, the methods disclosed herein further comprise administering to the subject a therapeutically effective amount of a cytosolic phospholipase A2 (cPLA2) inhibitor (including, for example, ASB-14780). In some examples, the methods disclosed herein further comprise administering to the subject a therapeutically effective amount of a solute carrier family 6 member 2 (SLC6A2) inhibitor (including, for example, reboxetine). In some examples, the methods disclosed herein further comprise administering to the subject an immune checkpoint inhibitor. Checkpoint inhibitors include, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

Also disclosed herein is a method of treating obesity or an obesity-associated disorder in a subject in need thereof comprising: obtaining a biological sample from the subject; determining that the subject has obesity or an obesity-associated disorder if there is an increased level of an S100A7 polypeptide in the biological sample in comparison to a reference control; and administering to the subject a therapeutically effective amount of a therapeutic agent to treat obesity or the obesity-associated disorder if the subject is determined to have obesity or an obesity-associated disorder.

In some embodiments, the S100A7 inhibitor is a polypeptide, a polynucleotide, a small molecule, or a gene editing system. In some embodiments, the polynucleotide is a siRNA or a shRNA that targets an S100A7 polynucleotide. In some embodiments, the gene editing system is a CRISPR/Cas endonuclease system. In some embodiments, the CRISPR/Cas endonuclease system comprises a guide RNA targeting an S100A7 polynucleotide (for example, an exon or a UTR of an S100A7 gene).

In some embodiments, the polypeptide is a recombinant antibody that specifically targets S100A7.

In some embodiments, the recombinant antibody comprises at least one heavy chain and/or at least one light chain. In some embodiments, the heavy chain comprises a heavy chain complementarity determining region 1(CDRH1), a heavy chain complementarity determining region 2(CDRH2), and/or a heavy chain complementarity determining region 3(CDRH3). In some embodiments, the CDRH1 comprises a sequence at least about 80% (for example, at least about 80%, about 85%, about 90%, about 95%, about 98%, or about 99%) identical to SEQ ID NO: 1 or a fragment thereof. In some embodiments, the CDRH2 comprises a sequence at least about 80% (for example, at least about 80%, about 85%, about 90%, about 95%, about 98%, or about 99%) identical to SEQ ID NO: 2 or a fragment thereof. In some embodiments, the CDRH3 comprises a sequence at least about 80% (for example, at least about 80%, about 85%, about 90%, about 95%, about 98%, or about 99%) identical to SEQ ID NO: 3 or a fragment thereof. In some embodiments, the heavy chain comprises a sequence of at least about 80% (for example, at least about 80%, about 85%, about 90%, about 95%, about 98%, or about 99%) identical to SEQ ID NO: 4 or a fragment thereof. The CDRH can comprise a sequence having a sequence identity percentage less than, greater than, or in-between the ranges mentioned above.

In some embodiments, the light chain comprises alight chain complementarity determining region 1(CDRL1), a light chain complementarity determining region 2(CDRL2), and/or a light chain complementarity determining region 3(CDRL3). In some embodiments, the CDRL1 comprises a sequence of at least about 80% (for example, at least about 80%, about 85%, about 90%, about 95%, about 98%, or about 99%) identical to SEQ ID NO: 5 or a fragment thereof. In some embodiments, the CDRL2 comprises a sequence of at least about 80% (for example, at least about 80%, about 85%, about 90%, about 95%, about 98%, or about 99%) identical to SEQ ID NO: 6 or a fragment thereof. In some embodiments, the CDRL3 comprises a sequence of at least about 80% (for example, at least about 80%, about 85%, about 90%, about 95%, about 98%, or about 99%) identical to SEQ ID NO: 7 or a fragment thereof. In some embodiments, the light chain comprises a sequence at least about 80% (for example, at least about 80%, about 85%, about 90%, about 95%, about 98%, or about 99%) identical to SEQ ID NO: 8 or a fragment thereof. The CDRL can comprise a sequence having a sequence identity percentage less than, greater than, or in-between the ranges mentioned above.

In some examples, the methods disclosed herein further comprise administering to the subject a therapeutically effective amount of a cytosolic phospholipase A2 (cPLA2) inhibitor (including, for example, ASB-14780). In some examples, the methods disclosed herein further comprise administering to the subject a therapeutically effective amount of a solute carrier family 6 member 2 (SLC6A2) inhibitor (including, for example, reboxetine). In some examples, the methods disclosed herein further comprise administering to the subject an immune checkpoint inhibitor. Checkpoint inhibitors include, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) or a combination thereof.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day. Dosages above or below the range cited above may be administered to the individual patient if desired.

Also disclosed herein are methods for diagnosing, detecting, or monitoring obesity or obesity-associated diseases (including, for example, cancers, autoimmune diseases, or fatty liver disease) in a subject comprising: obtaining a biological sample from the subject; and determining that the subject has obesity or an obesity-associated disorder if there is an increased level of an S100A7 polypeptide in the biological sample in comparison to a reference control.

Also disclosed herein are methods for monitoring a therapeutic for obesity or obesity-associated diseases (including, for example, cancers, autoimmune diseases, fatty liver disease) in a subject comprising obtaining a biological sample from the subject, wherein the subject was and/or is treated with the therapeutic; and determining that the therapeutic is effective to treat obesity or an obesity-associated disorder if there is a decreased level of an S100A7 polypeptide in the biological sample in comparison to a reference control (e.g., levels of S100A7 prior to the treatment).

### Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Example

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: S100A7 and Obesity-Associated Disorders

Chronic inflammation, a well-known cause of cancer, is also a central attribute of obesity. S100A7 is an anti-microbial pro-inflammatory protein, and its biological role has been reported in different human chronic inflammatory conditions such as psoriasis and breast cancer. Studies have shown that S100A7 mediates its tumorigenic effects through RAGE. Importantly, genetic ablation of the receptor for advanced glycation end-products (RAGE) protects the mice from high-fat diet (H1FD)-induced obesity. However, the functional role of S100A7 itself in obesity and obesity-associated breast cancer remains elusive. Therefore, the expression of S100A7 was first evaluated in HFD-induced and genetically obese mouse models, and surprisingly, both genetically and HFD-induced obese mice models were observed to express a high level of S100A7 as compared to their respective experimental controls. These observations provided the rationale to generate the S100A7 whole-body knockout mouse model to understand HFD-induced obesity and obesity-associated breast cancer. In this example, the whole-body deletion of S100A7 prevented HFD-induced obesity. This is the first evidence that S100A7 inhibition protects mice from HFD-induced obesity by modulating metabolic and molecular changes associated with adiposity and mitochondrial functions. Interestingly, the genetic ablation of S100A7 also suppresses breast tumor growth in-vivo. Importantly, an S100A7 neutralizing antibody (nAb) was also developed to directly target the S100A7 in obesity and breast cancer. In-vitro studies revealed that S100A7 nAb inhibits the S100A7-mediated migration, invasion, and expression of PD-L1 in triple-negative breast cancer (TNBC) cells. In addition, S100A7 nAb in combination with a cPLA2 inhibitor was discovered to synergistically inhibit the S100A7-mediated breast tumor growth and metastasis. Finally, the efficacy of S100A7 nAb was evaluated on HFD-induced obesity and breast cancer. This study revealed that S100A7 nAb alone or in combination with FDA-approved SLC6A2 inhibitor significantly reduced HFD-induced obesity as well as obesity-associated breast tumorigenicity. In brief, S100A7 plays a crucial role in obesity and breast cancer and its targeting can be very effective in treating several human chronic inflammatory conditions including obesity and obesity-associated breast tumorigenesis.

Over 60% of the adult population is considered overweight (body-mass-index (BMI) of 25-29.9) and 38% of the population is considered obese (BMI≥30) in the USA alone and this is especially true in women of African descent who manifest a 58.6% obesity rate. Additionally, breast cancer patients with pre-existing obesity-associated diabetes are at higher risk for distant metastasis and premature mortality. Obesity, while a complex phenotype, is closely associated with inflammation and the availability of several growth factors that support tumor growth. Triple-negative breast cancer (TNBC), which represents approximately 20% of all breast cancer cases, is closely associated with a worse prognosis and low survival due to early metastasis. Epidemiological data have shown a strong association between obesity and TNBC initiation and progression. Moreover, obesity increases 30-50% TNBC associated mortality risks. Few studies also show that TNBC is strongly associated with obesity, metabolic syndrome, and younger age of incidence. Identifying mechanisms and therapeutic targets that link obesity with TNBC and metastasis contributes to developing better therapies for these patients.

It has been well known that chronic inflammation plays an important role in causing obesity and obesity-associated cancers. S100A7, a pro-inflammatory protein, was initially observed to be highly expressed in overweight females as compared to normal-weight women. S100A7 is also shown to be highly expressed in TNBC patients as compared to non-TNBC subjects. S100A7 (Psoriasin) was first discovered as a small low-molecular-weight (11.4 kDa) cytoplasmic protein and described as a member of the S100 gene family, which is positioned within the S100 gene cluster on 1q21 chromosomal locus. This secreted inflammatory protein possesses the typical calcium-binding domains and is isolated from keratinocytes of psoriasis patients.

The abnormal expression of S100A7 is associated with different chronic inflammatory conditions such as auto-immune diseases and SARS-CoV-2 infection. It has also been reported that S100A7 can be expressed by cancerous cells and the report described its abnormal role in the progression and differentiation of the cell cycle. The abnormal function of this protein is also supported in different human malignancies apart from its role in chronic inflammatory autoimmune diseases. The latest findings show that the copy number of the chromosomal region containing S100A genes, including S100A7, gets amplified in subpopulations of cancer stem cells (CSCs), especially in TNBC patients. The high expression of S100A7 protein has been reported in tumors from numerous tissues such as the breast, skin, bladder, head and neck, lung, and stomach, with a robust correlation to the worst prognosis. In pathological situations, S100A7 is secreted into the extracellular compartment, where it can induce the migration of tumor cells and immunosuppressive cells. Furthermore, the overexpression of this protein is shown in an in-vivo transgenic mice model with augmented breast tumor growth and metastasis. It is also known to enhance the proliferation of endothelial cells, through its interaction with the RAGE receptor. Remarkably, it was also revealed that the inhibition of the S100A7-mediated oncogenic signaling through the suppression of RAGE leads to reduced lung metastasis in breast cancer. Interestingly, RAGE-mediated adipose tissue inflammation and insulin signaling have also been reported as essential mechanisms that contribute to the development of obesity-associated insulin resistance.

Obesity and TNBC are highly prevalent in African American (AA) ethnicity. Surprisingly, it was also indicated that AA TNBC women showed a significantly higher level of S100A7 as compared to their Caucasian counterparts. It has been shown that S100A7 overexpression increases the growth and metastasis of TNBC cells, while its down-regulation inhibits the growth and metastasis of TNBC cells. However, nothing is known about the role of S100A7 in obesity and obesity-associated breast cancer, especially in TNBC. Phylogenetic analyses have shown the mouse ancestor mS100A7/A15 to be most related to S100A7 and S100A15 among the human paralogs. Like human S100A7, Jab-1 motif is also present in mS100a7a15 but is absent in human S100A15. Therefore, the MMTV-rtTA/TetO-mS100a7a15 bi-transgenic mice that overexpress mS100a7a15 (mS100a7a15-OE) in mammary glands (MGs) was generated. It has been shown that the overexpression of mS100a7a15 induces hyperplasia in MGs of these transgenic mice and enhanced tumor growth and metastasis in the syngeneic mouse model.

Since mammary-associated adipocytes form a substantial part of MG, there is some crosstalk between MGs and the adipose tissue (AT) which enhances breast tumorigenesis. To further elucidate the role of S100A7 in obesity-associated breast cancer, a mS100a7a15 floxed mouse and whole body mS100a7a15 knockout (S100A7 KO) mouse were generated. Astonishingly, whole-body deletion of S100A7 drastically inhibits breast tumor burden in S100A7 KO mice injected with murine TNBC cell line. Interestingly, the results indicated that the S100A7 KO mice showed a protective effect against high-fat diet (HFD)-induced obesity and showed an increased presence of multilocular lipid droplets indicative of beiging, or increased presence of brown-like adipocytes in WAT. BAT is an important tissue with regard to glucose metabolism and insulin sensitivity. BAT is a powerful tool to combat obesity because it can expend chemical energy in the form of heat. It has also been demonstrated that increasing the amount of BAT using a transplantation model improved whole-body metabolic health and increased insulin-stimulated glucose uptake.

It has also been found that antibody-based therapeutics against human malignancies and cancer are exceedingly effective in the clinic and currently enjoy unparalleled recognition of their potential. So far, 13 mAbs have been approved for clinical use in the United States and the European Union. The monoclonal S100A7 nAb was also developed and its efficacy tested for inhibiting the TNBC tumorigenicity. The S100A7 nAb in combination with another therapeutic regimen showed promising effects against S100A7-mediated breast cancer, especially TNBC. S100A7 neutralization alone or in combination with other drug was noted to reduce HFD-induced obesity and breast tumor burden. Therefore, S100A7 targeting using this specific nAb is an effective therapeutic strategy to inhibit the S100A7-induced pathophysiological chronic inflammatory conditions including obesity and breast cancer.

### Example 2: Material and Methods

Cell culture and other reagents. Human breast carcinoma cell line MDA-MB-231 was obtained from ATCC. Mvt-1 cells were cultured as described earlier. S100A7 overexpressing MDA-MB-231 stable clones were used. Dulbecco's modified eagle medium, fetal bovine serum, penicillin and streptomycin antibiotics, trypsin, and ethylenediaminetetraacetic acid were obtained from Gibco BRL (Grand Island, NY, USA). RAGE Antagonist (FPS-ZM1) and recombinant S100A7 were purchased from Calbiochem and Novus Biologicals respectively. ASB-14780 and reboxetine were purchased from Sigma-Aldrich.

Western blotting, adipokine array and Immunohistochemistry (IHC). Cell or tissue lysates were analyzed by Western blotting. Briefly, samples were run on NuPAGE 4-12% gradient precast gels (Invitrogen) and probed with primary antibodies (Table 1), followed by secondary antibodies incubation, and were developed. Adipokine array on mouse blood samples was performed as per manufactures protocol (R & D Systems). IHC on formalin-fixed sections was performed. The antibodies used are mentioned in Table 1.

qRT-PCR and ELISA. Total RNA was isolated from mouse tissue samples using RNeasy Mini Kit (Qiagen) and cDNA was prepared using High-Capacity cDNA Reverse Transcription Kit (Thermo Fisher). The expression level of mS100a7a15 was determined using specific primers (Table 2) and SYBR™ Green PCR Master Mix (Applied Biosystems, Foster City, CA, USA). The expression level of leptin gene was determined by using specific TaqMan™-based probes and primers manufactured by Thermo Fisher. Human S100A7 ELISA kits were purchased from the Novus Biologicals and ELISA was performed.

Generation of mS100a7a15 (S100A7) floxed and whole-body knock-out (KO) mice model. S100A7 conditional KO mice were generated by the Ohio State University Genetically Engineered Mouse Modeling Core using standard embryonic stem cell (ES) homologous recombination technology. Briefly, the targeting vector PG00263_Y_1_A03 “conditionally ready” (tm1a allele) was obtained from the International Knockout Mouse Consortium (project number 119798). After sequencing, the vector was linearized by AsiI restriction digest and electroporated into S1B6a, a mixed genetic background 129Sv-C57Bl/6 mouse embryonic stem cell line. G418-resistant clones were selected and screened by southern blotting with probes specific to regions of the S100A7 locus outside the 5′ homology arm of the targeting vector. Correctly targeted ES clones were used to generate chimeras that transmitted the mutated allele to the germ line. For experimental purposes, mice were crossed first to a Flpe ubiquitous line (Jackson Laboratories strain number: 005703) to remove the LacZ-Neo cassette flanked by frt sites and obtain the clean conditional allele (tm1c). Mice were subsequently backcrossed onto the FVB/NJ strain background for up to 10 generations to develop pure lines of mS100a7a15^{F/F} (S100A7 floxed) mouse model. To analyze the effect of S100A7 deletion from the whole body, S100A7 floxed mice were bred with Sox2-Cre (Jackson Laboratory) to generate mS100a7a15^{F/F}; Sox2-Cre (S100A7 KO). Mice genotypes were identified by PCR analysis (Table 2). Animals were allowed to reach adulthood and euthanized the mice at the age of 20 weeks. The whole-body deletion of S100A7 was determined using qRT-PCR, IHC, and Western blotting.

Analysis of HFD-induced obesity in S100A7 floxed and KO mice. Mice were fed a normal diet or HFD (60% kcal from fat; 1.17% total Pi, Research Diets Inc., New Brunswick, NJ, USA) for 14 weeks. Mice were fed ad libitum. Body weight and food intake were also analyzed. All the mice were sacrificed at approximately 19 weeks of age. All the organs including white, brown, and subcutaneous adipose tissues were harvested, fixed in 2% paraformaldehyde, and stained with hematoxylin and eosin (H/E) with the assistance of OSU comparative pathology & digital imaging shared resource (CPDISR). Oiled red-O staining was also performed on fresh frozen OCT embedded liver tissues with the assistance of OSU CPDISR facility. Insulin (ITT) and glucose (GTT) tolerance tests were performed.

### Development and Sequencing of Human S100A7 nAb.

Roller Bottle Antibody Production and Protein AG Purification. Below are the Following Steps for the Production and Purification of Human S100A7 nAb:
1. Culture the hybridomas with DMEM medium containing 10% FBS and incubate at 37° C., 5% CO₂ in a humidified incubator. Prepare roller bottle cell culture medium by FEM medium. When hybridoma cells are at >70% confluence, transfer the hybridoma cells suspension to shake flask culture flask containing roller bottle cell culture medium.
2. Incubate the shake flask culture flask on the humidified table at 37° C., 5% CO₂. Test the cell supernatant with ELISA assay when hybridoma cells are at >70% confluency.
3. Count cells with hemacytometer 2 days later. Prepare hybridoma cell suspension with roller bottle cell culture medium. Transfer roller bottle cell culture medium and hybridoma cell suspension to tissue culture roller bottle.
4. Incubate the tissue culture roller bottle in an electro-thermal incubator for around 7 days. Count the cells with a hemacytometer. Collect cell supernatant on the 7th day. Centrifuge the cell suspension at 8000 rpm for 20-30 minutes to collect the supernatant. Filter the supernatant with a 0.22 m filter to remove debris.
5. Completely suspend Protein A/G resin in the bottle and transfer the required amount of the resin to a clean empty column. Allow the resin to settle down and the buffer to drain from the column.
6. Add 10 resin volumes of the Binding/Wash Buffer onto the column to equilibrate the resin. Transfer the Antibody sample to the column and collect flow-through for the test.
7. Connect the column with a nucleic acid protein detector by silicone tube and turn on the power to monitor the absorbance of the effluent at A280. Add Binding/Wash Buffer onto the column to wash resin until the absorbance at A280 is stable.
8. Allow the Binding/Wash Buffer to drain from the column and add the Elution Buffer to the resin to elute the antibody bound to the resin. Collect the eluate containing the antibody in a tube with the assistance of a Nucleic acid protein detector.
9. Immediately add the Neutralization Buffer to the tube containing antibody eluate to neutralize pH. To regenerate resin, add 3 resin volumes of the Regeneration Buffer to the column followed by equilibration with 10 resin volumes of the Binding/Wash buffer.
10. Transfer the antibody eluate to a dialysis bag and dialyze it against PBS at 2-8° C. overnight. Add Sodium Azide Stock Buffer to the antibody solution with the final concentration of 0.02% and store at 2-8° C.

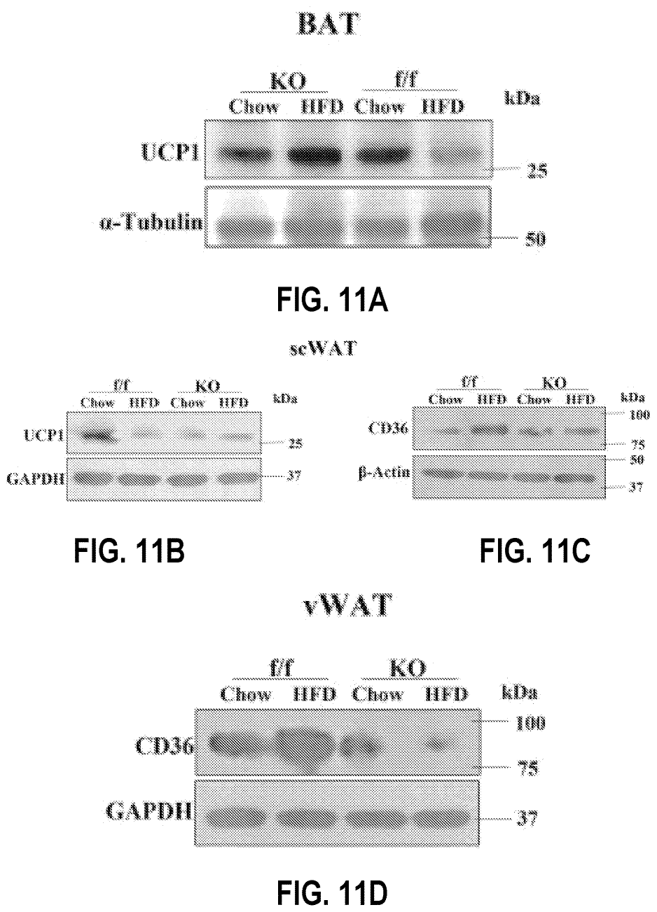

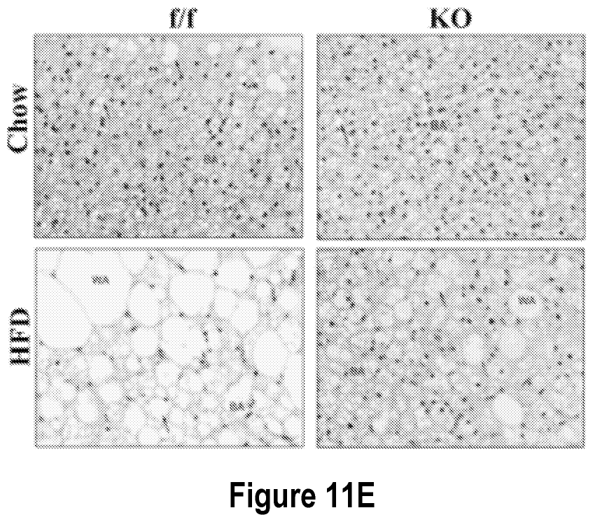

Antibody Full-Length Sequencing of Hybridoma 28F8-2. Below are the following steps for the production and purification of human S100A7 nAb: Total RNA was isolated from the hybridoma cells following the technical manual of RNA-easy Isolation Reagent. Total RNA was then reverse transcribed into cDNA using isotype-specific anti-sense primers or universal primers following the technical manual of SMARTScribe Reverse Transcriptase. The antibody fragments of VH and VL were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of the correct sizes. No less than five colonies with inserts of correct sizes were sequenced for each fragment. The sequences of different clones were aligned, and the consensus sequence of these clones was provided (FIG. 11).

Mouse models, obesity tumor growth analyses and flow cytometry. The NCG and wild-type 6 weeks old female FVB mice (6 weeks old) were obtained from the Charles River. S100A7 overexpressing MDA-MB-231 cells (1×10⁶) were injected into the 4^{th} mammary fat pad of NCG mice. While Mvt1 cells (1×10⁵) were injected into the mammary fat pad of wild type FVB mice fed with HFD. After the onset of palpable tumors, mice bearing Mvt1 tumors were treated with vehicle control (VC), S100A7 nAb (100 g/mouse; i.p. route; clone 28F8-2; two times a week) alone or reboxetine (20 mg/kg·bt, i.p.; twice a week) alone or in combination for up to 20 weeks. Mice bearing S100A7 overexpressing MDA-MB-231 tumors were either treated with vehicle control (VC), S100A7 nAb (100 g/mouse, i.p. route; clone 28F8-2; two times a week) alone or in combination with ASB-14780 (50 mg/kg·bt, oral route; three times a week). Mvt-1 cells (1×10⁵) were also injected into the mammary glands of S100A7 floxed (control) and KO mice. Body weights and tumors were measured weekly, and volume was calculated. In the endpoints, animals were sacrificed, and tumors and other organs were excised. All mice were kept in The OSU's animal facility in compliance with the guidelines and protocols approved by the OSU-IACUC. For flow cytometry, freshly prepared single-cell suspensions of spleen samples harvested from non-tumor bearing S100A7 floxed and KO mice were incubated with an Fc receptor blocker followed by staining with different fluorochrome-conjugated antibodies (Table 1). After staining, cells were evaluated by FACS Fortessa using CellQuest software (BD Biosciences)

Trans-well migration assay and 3D-Spheroid invasion assay. MDA-MB-231 cells were treated with hS100A7 neutralizing antibody (clone 28F8-2; 300 ng/μl) or control IgG or PBS in the presence or absence of recombinant human S100A7 (100 ng/ml). Migration assays were performed overnight using the treated cells as described above utilizing transwell chambers (Costar 8 m pore size). For the 3D-Spheroid invasion assay, 1×10³ cells were seeded with extracellular matrix (ECM) in round bottom ultra-low attachment plates for 7 days in the presence or absence of the above treatments followed by the addition of invasion matrix and treatment regimens. On the 10^{th} day, the invaded area was calculated by using the image-J plugin.

Statistical Analysis. For continuous variables, two-sample t-tests were used if two groups were compared, and One-way ANOVA was used when more than two groups were compared. Non-parametric tests were used to calculate the p values for comparing the human data.* indicates P<0.05; ** indicates P<0.01; *** indicates P<0.001; **** indicates P<0.0001; ns is non-significant.

### Example 3: Results

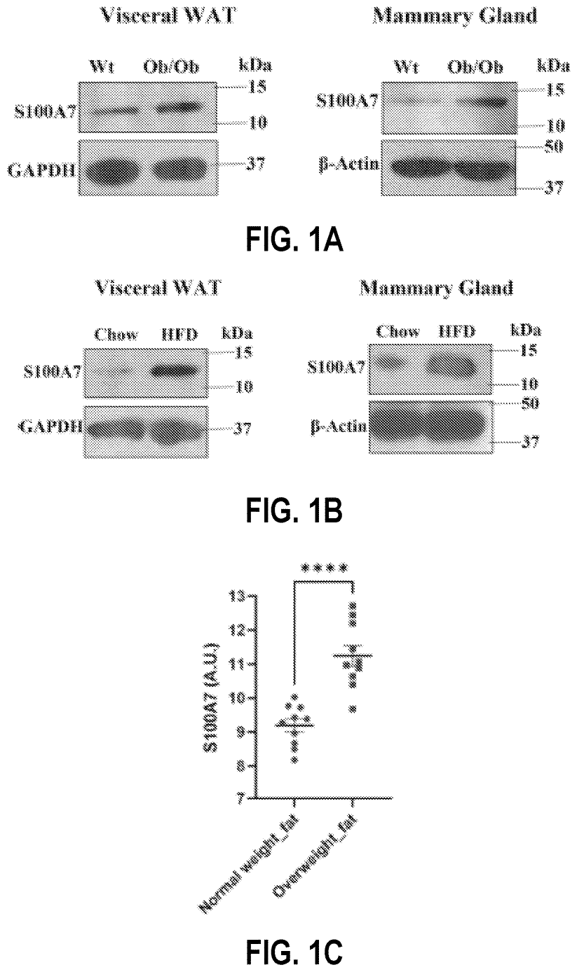

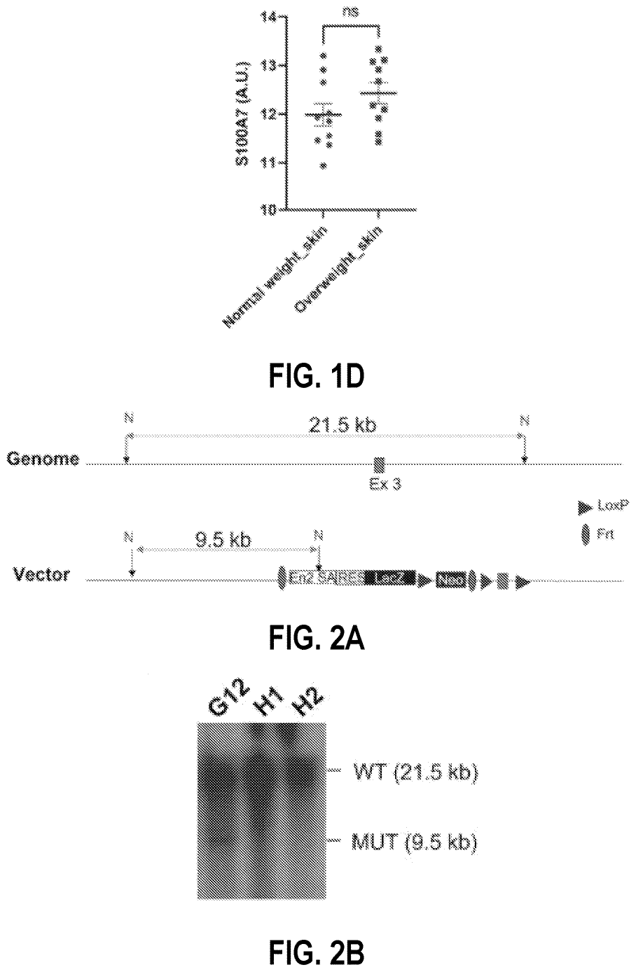

Expression of S100A7 in obesity. Although obesity has been directly correlated with TNBC-related mortality, the molecular link between obesity and TNBC has not been established. It has been shown that S100A7 downregulation inhibits, and S100A7 overexpression enhances TNBC cell growth in vitro. It has been further shown that in an orthotopic mouse model of breast cancer, S100A7 overexpression enhances tumor growth of TNBC cells. Previously, it has been reported that RAGE plays an essential role in obesity and metabolic alterations. Furthermore, it has been shown that S100A7 mediates its effect through RAGE in breast cancer cells. Although, the expression and functional role of S100A7 itself in obesity remain largely unknown. In this example, the expression of S100A7 was first evaluated in MGs and visceral white adipose tissue (vWAT) samples harvested from age-matched wild-type normal weight and B6·Cg-Lep^{ob}/J obese female mice. B6·Cg-Lep^{ob}/J obese mice were observed to have expressed a higher level of S100A7 in MGs and vWAT as compared to same-aged normal weight wild type mice (FIG. 1A). A higher level of S100A7 was also observed in MGs and vWATs harvested from HFD-induced obese mice as compared to mice provided with normal chow (FIG. 1). Interestingly, a significantly increased expression of S100A7 was also observed in fats harvested from overweight women as compared to normal-weight females using GSE151839 GEO dataset (FIG. 1C). However, any significant increase in S100A7 expression was not observed in skin samples of overweight females using the same dataset (FIG. 1D).

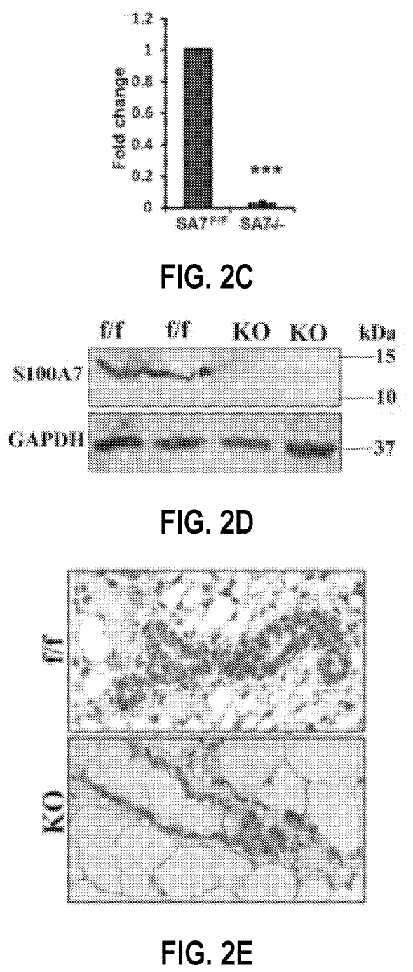

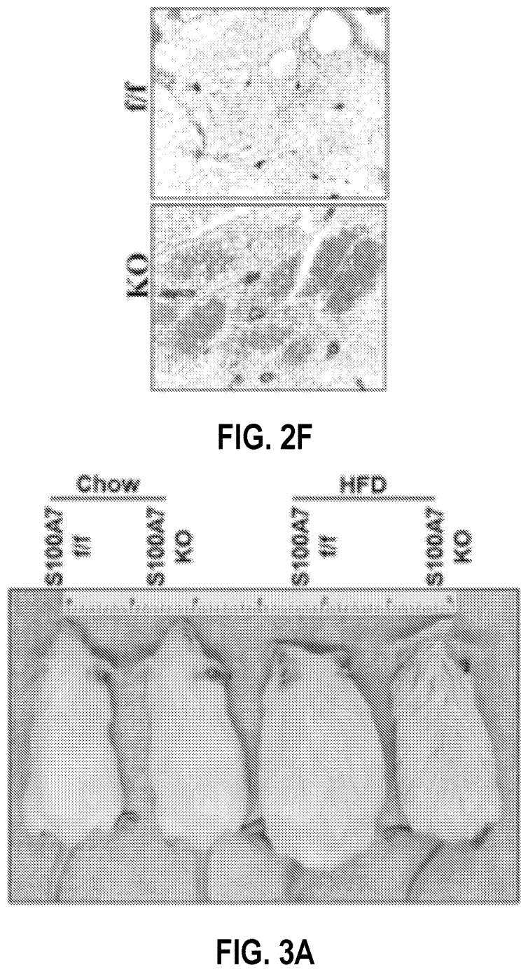

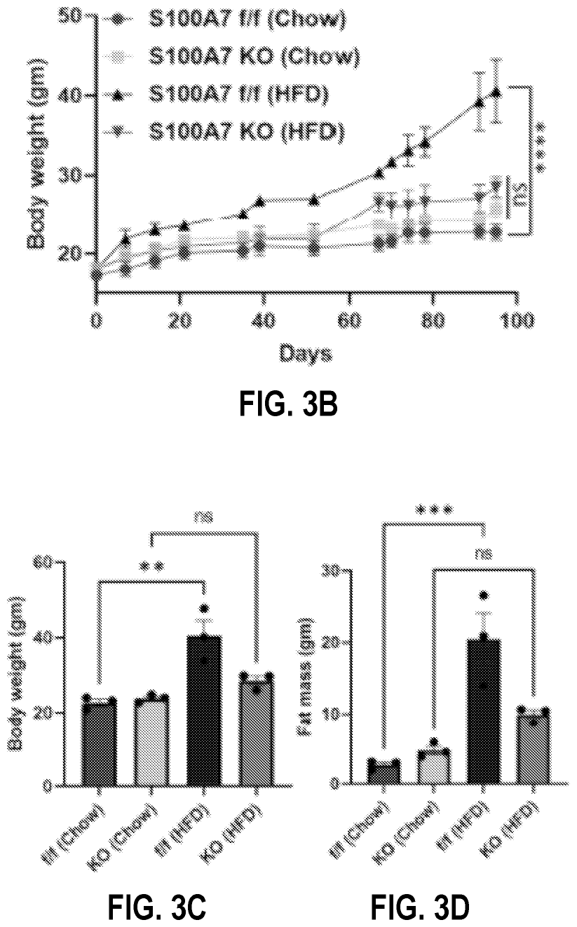

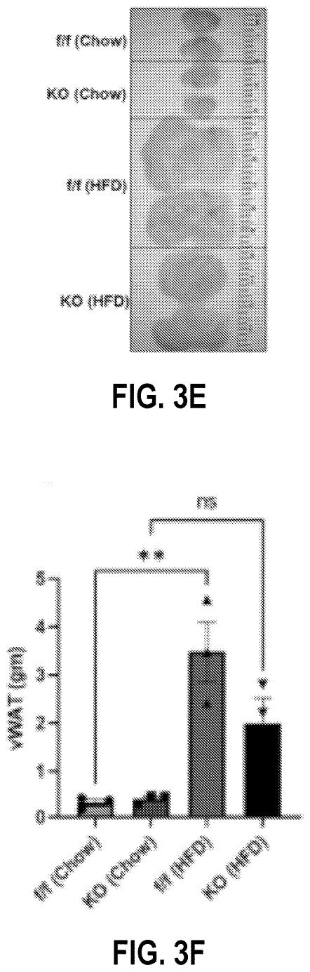

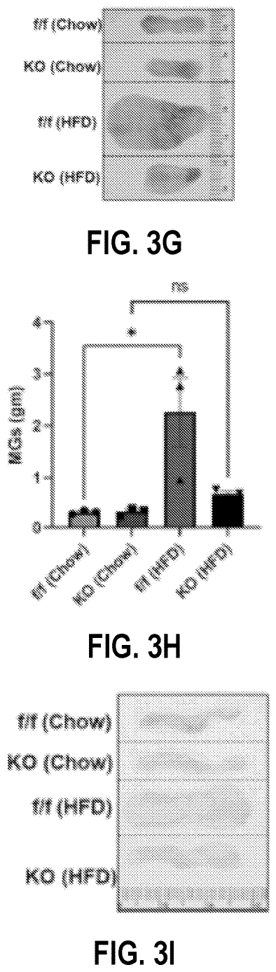

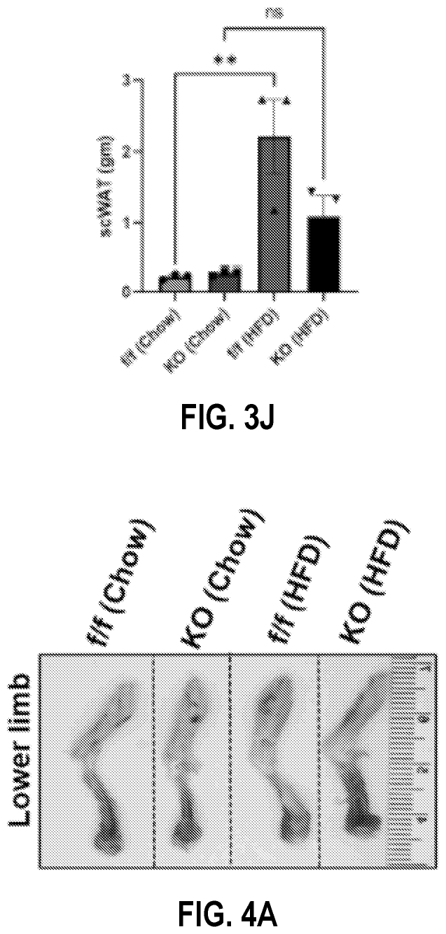

Generation of S100A7 whole body knock-out mouse for the evaluation of HFD-induced obesity. Since S100A7 is found to be highly expressed in overweight or obese pathophysiological conditions, it was deleted in mice from the whole body by the OSU Genetically Engineered Mouse Modeling Core Facility using standard embryonic ES homologous recombination technology (FIGS. 2A and 2B). To analyze the effect of S100A7 deletion from whole body, S100A7 floxed mice were bred with Sox2-cre (Jackson Laboratory) mice to generate mS100a7a15^{f/f}; Sox2-Cre (S100A7 KO). Animals were allowed to reach adulthood and the mice were euthanized at the age of 20 weeks. WB, IHC and qRT-PCR analysis confirmed the successful deletion of the S100A7 gene from the whole body (FIGS. 2C, 2D, and 2E). Upon histological examination of S100A7 floxed and KO mice, a reduced size and increased presence of multilocular lipid droplets resembling a beige AT phenotype was observed (FIG. 2F). Beige or brite (brown in white) adipocytes are a particular type of adipocyte and have multilocular lipid droplets, a central nucleus, and a high density of mitochondria, similar to brown adipocytes. Beige adipocytes function similarly to brown adipocytes in that they directly generate energy in the form of heat, contributing to thermogenesis, measured as an increased core body temperature. The effect of HFD treatment was also analyzed in causing obesity in S100A7 floxed and KO mice. Surprisingly, S100A7 KO mice were protected from HFD-induced body weight gain (FIGS. 3A, 3B, and 3C), and importantly, the lower body weight in S100A7 KO mice was predominantly featured by the reduction of fat mass as determined by echoMRI (FIG. 3D).

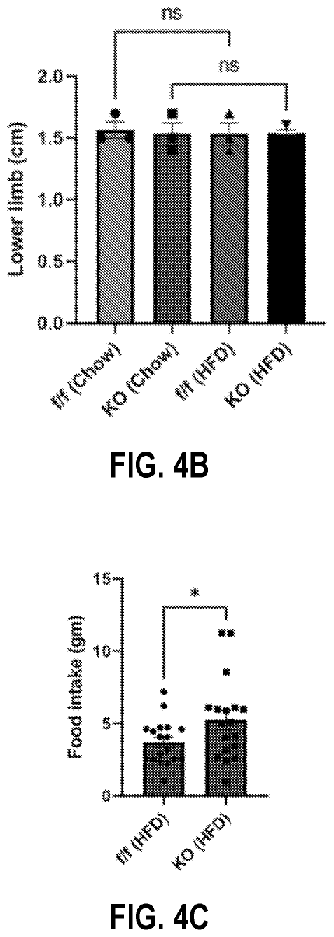

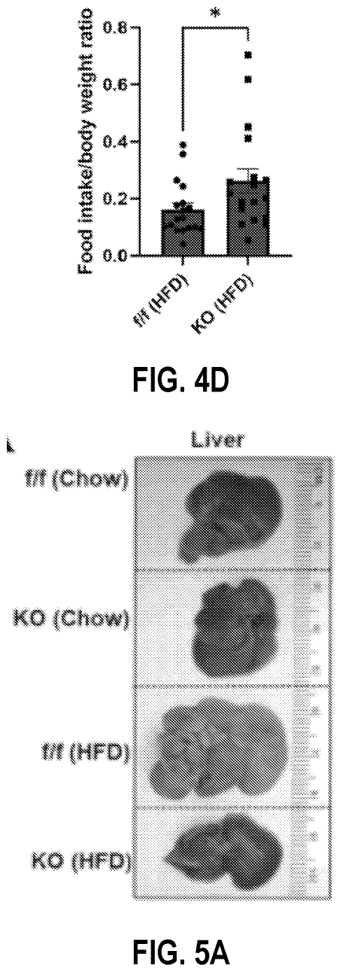

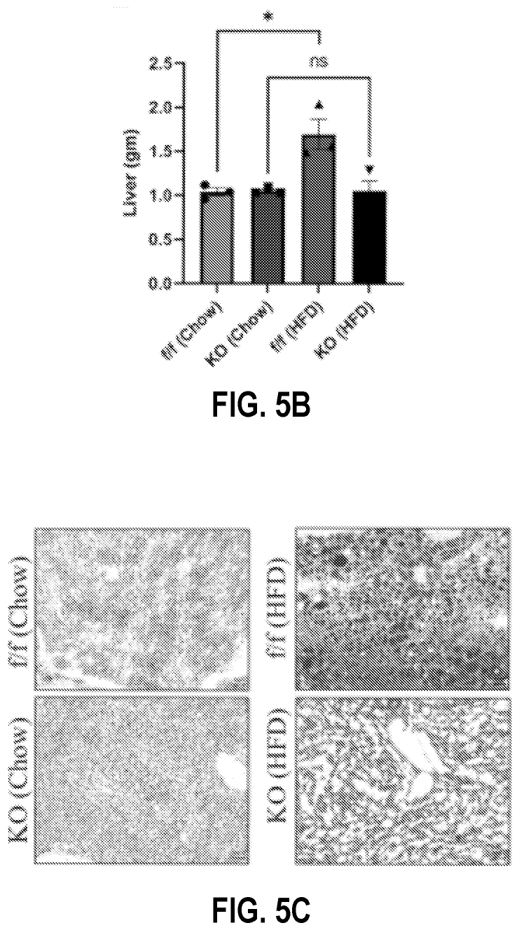

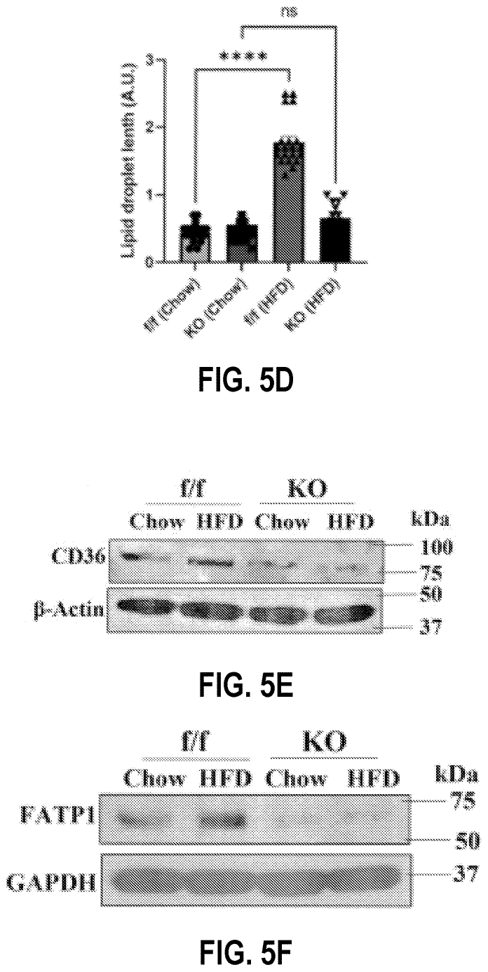

Effect of S100A7 whole body deletion on food intake, accumulation of WATs, and morphological changes in the liver. In this example, S100A7 KO mice were protected from HFD-induced increase in the overall weight of mammary glands, and visceral and subcutaneous WATs (FIGS. 3E, 3F, 3G, 3H, 3I, and 3J). Of note, an insignificant difference in tibia length, a marker for mouse development, between floxed and KO mice was observed (FIGS. 4A and 4B), showing that the lower body weight identified in S100A7 KO mice was not caused by the growth retardation. Furthermore, the measurement of food intake revealed that S100A7 KO mice consumed more food than their floxed mice counterparts (FIGS. 4C and 4D). Moreover, S100A7 deletion also protects the mice from HFD-associated fatty liver as compared to control floxed mice (FIGS. 5A and 5B). The accumulation of Triglycerides (TGs) was also examined in the liver of floxed, and KO mice fed either with chow or HFD using Oil-Red O staining. The liver of KO mice showed an insignificant increase in HFD-induced accumulation of TGs as compared to KO mice fed with chow (FIGS. 5C and 5D). However, the accumulation of TGs in liver was significantly higher in floxed mice provided with HFD relative to floxed mice fed with chow (FIGS. 5C and 5D).

The high expression of CD36 and FATP1 has been reported as well-known markers associated with lipid storage in diet-induced obesity. Therefore, their expression in the above groups was also assessed. It was found that the treatment of HFD in floxed mice increased the expression of both these proteins as compared to floxed mice provided with chow (FIGS. 5E and 5F). However, no noticeable change in the expressions of these proteins was observed in liver tissues of KO mice fed with HFD as compared to KO mice provided with chow (FIGS. 5E and 5F).

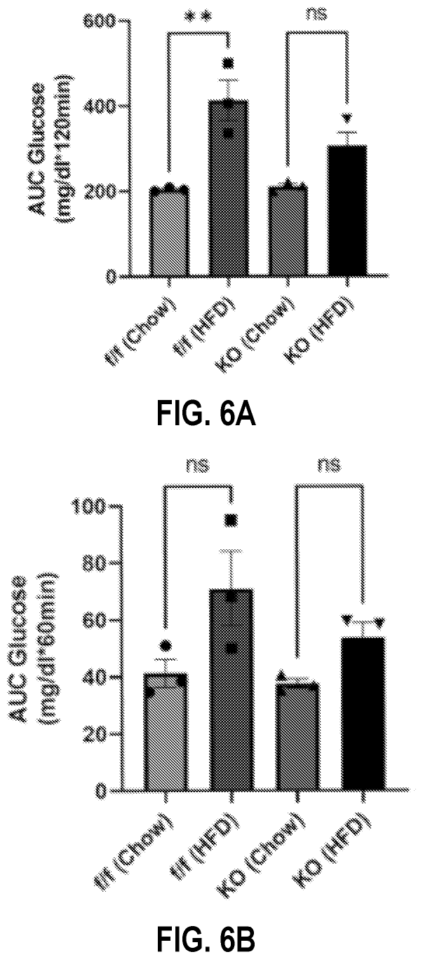

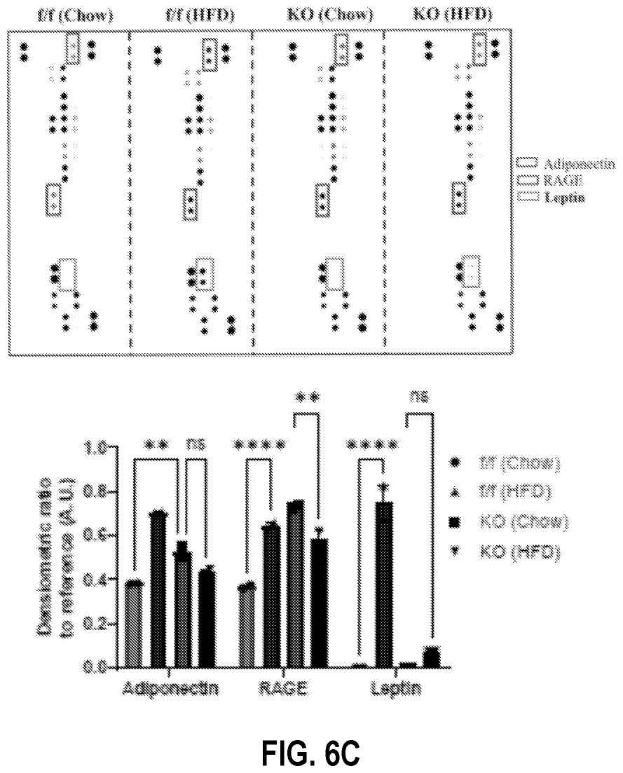

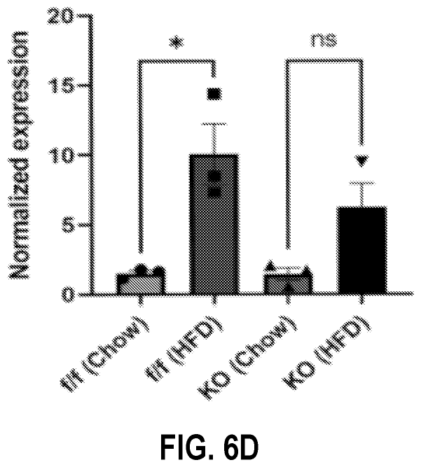

Effect of S100A7 whole body deletion on HFD-induced metabolic and molecular alterations. The effect of S100A7 deletion on HFD-induced metabolic alterations was evaluated by performing glucose tolerance tests (GTT) and insulin tolerance tests (ITT). GTT analysis revealed that only S100A7 KO mice are protected against the HFD-induced worsening in glucose tolerance (FIG. 6A), while floxed mice were unable to control their HFD-induced worsening in glucose tolerance (FIG. 6A). Although, no significant protection of S100A7 KO mice was observed against HFD-induced worsening of insulin tolerance in ITT (FIG. 6B).

Next, the level of secreted adipokines was determined in blood plasma samples of S100A7 floxed and KO mice fed with chow or HFD. Among all the adipokines, leptin was significantly elevated in the blood plasma sample of floxed mice provided with HFD as compared to floxed mice fed with chow (FIG. 6C). Interestingly, there was no significant change in the level of leptin in the plasma sample of KO mice fed with HFD compared to KO mice provided with chow (FIG. 6C). Therefore, the effect of HFD was also validated on gene expression of leptin in vWATs using TaqMan-based qRT-PCR. In agreement with the adipokine array result, a similar expression pattern of leptin gene was observed in floxed, and KO mice fed either with chow or HFD (FIG. 6D).

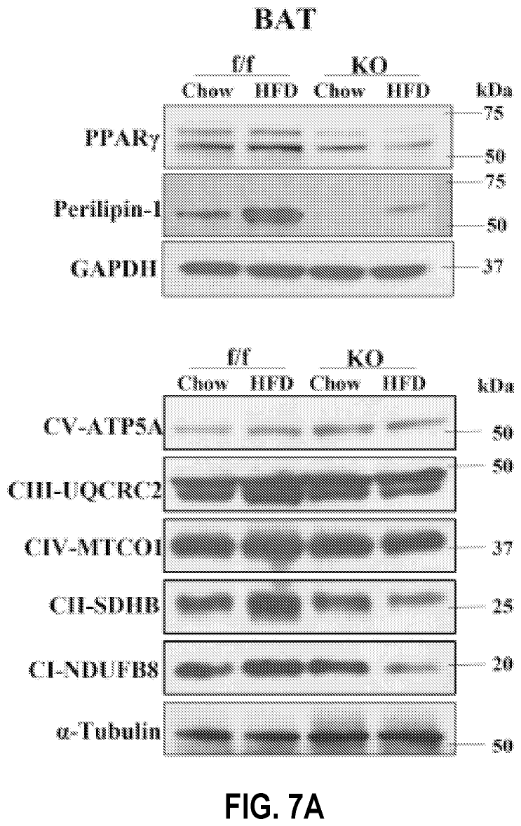

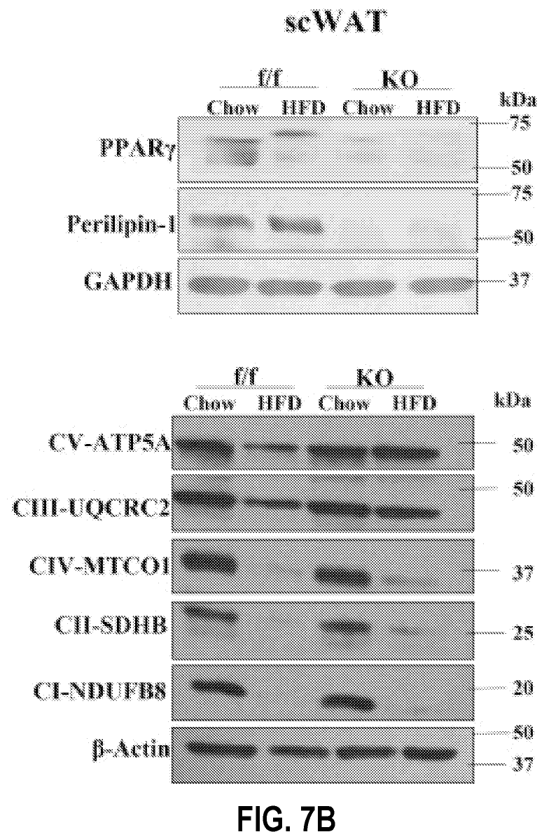

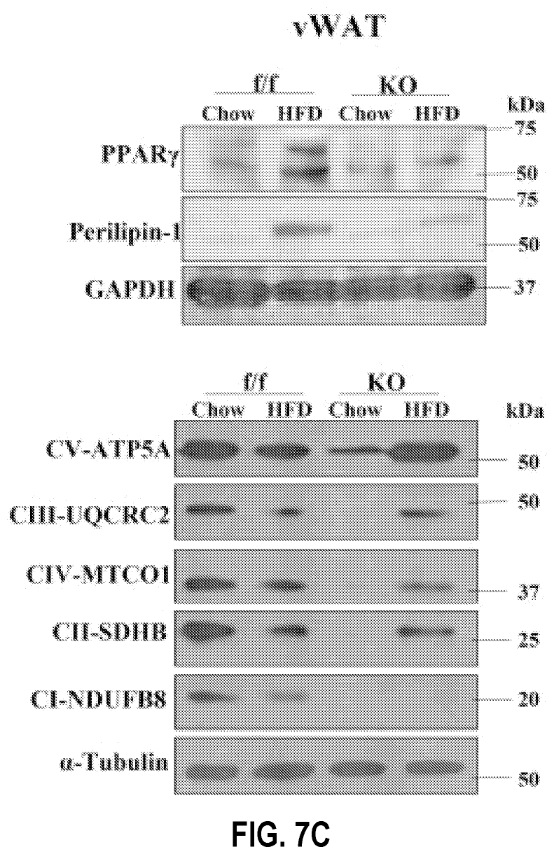

Effect of S100A7 whole body deletion on HFD-induced changes in expression of PPARγ, perilipin, UCP1, and mitochondrial oxidative phosphorylation complexes in different fat depots. It has been reported that the deletion of PPARγ in adipose tissues of mice protects against HFD-induced obesity and obesity-associated metabolic alterations. Furthermore, perilipin inhibition protected the mice against HFD-induced obesity, inflammation in adipose tissues, and the development of fatty liver disease. Therefore, the effect of deleting S100A7 from the whole body was examined on the diet-induced expression of PPARγ and perilipin-1 in different fat depots. S100A7 deletion suppressed the HFD-induced upregulation of PPARγ and perilipin-1 in BAT, vWAT and scWAT (FIGS. 7A, 7B, and 7C).

Mammals have two types of thermogenic adipocytes: brown and beige adipocytes. Thermogenic adipocytes have been shown to express higher levels of uncoupling protein 1 (UCP1) to dissipate energy in the form of heat by uncoupling the mitochondrial proton gradient from mitochondrial respiration. It has also been reported that UCP1 is the center of BAT thermogenesis and systemic energy homeostasis. Importantly, the overexpression of UCP1 either genetically or pharmacologically has been shown to inhibit obesity and improve overall insulin sensitivity. Surprisingly, S100A7 deletion also inhibits the HFD-induced downregulation of UCP1 in BAT and scWAT (FIGS. 11A and 11B). In addition, S100A7 ablation inhibits the HFD-mediated upregulation of CD36 in scWAT and vWAT (FIGS. 11C and 11D). CD36 has been known as a marker of human adipocyte progenitors with pronounced TGs accumulation and adipogenesis abilities. Interestingly, it was also revealed that the severity of lipid droplet hypertrophy in BAT was higher in floxed mice compared to S100A7 KO mice fed with HFD, with a few more normal brown adipocytes visible (FIG. 11E).

Finally, the effect of S100A7 ablation on HFD-induced changes in the expression of five different mitochondrial oxidative phosphorylation complexes was measured in different fat depots. Importantly, mitochondria are very important for the development of obesity because it integrates different metabolic information, such as ATP levels, oxidative stress, cell signaling, inflammation, and ER stress, which play crucial roles in the onset and maintenance of obesity. Astonishingly, S100A7 deletion reduced the HFD-induced overexpression of complex-II (SDHB) in BAT (FIG. 7A). SDHB deficient mice have been shown to be completely resistant to HFD-induced obesity. It was also observed that the decreased expression of complex-I in BAT harvested from S100A7 deleted mice fed with HFD (FIG. 7A). However, no changes were observed in the expression of complex-I and II in scWAT of S100A7 deleted mice fed with HFD (FIG. 7B). Whereas S100A7 deletion was found to inhibit the HFD-induced downregulation of complexes-III (UQCRC2), and V (ATP5A) in scWAT (FIG. 7B). The high-intensity exercise is very effective for the browning of WAT through the increased expression of complex-V and UCP1. In addition, the low expression of complexes-III and V were also noted in overweight individuals as compared to normal counterparts. S100A7 genetic ablation also inhibits the HFD-induced reduced expression of all the four mitochondrial oxidative phosphorylation complexes except complex I in vWAT (FIG. 7C). In brief, S100A7 whole-body deletion inhibits HFD-induced obesity by modulating the expression of different markers associated with adipogenesis and mitochondrial functions.

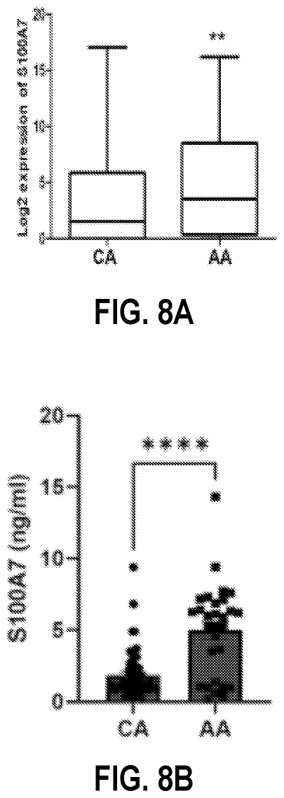

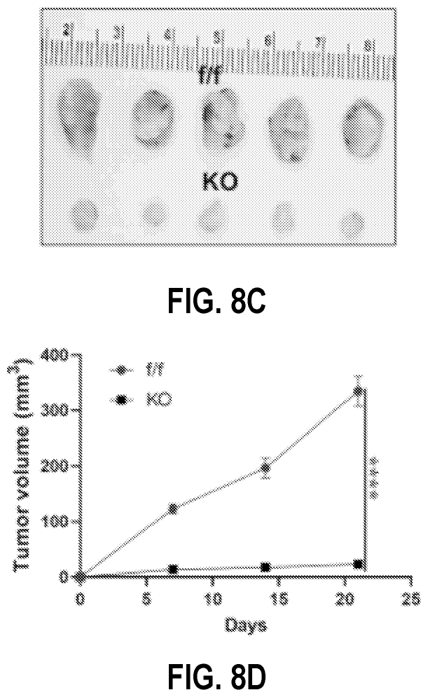

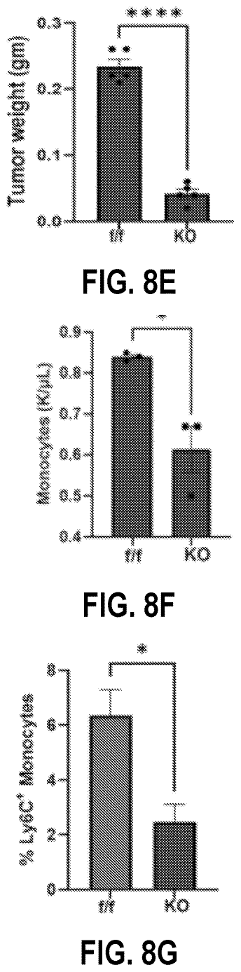

Effect of S100A7 global deletion on breast tumor growth and monocytes abundance. As obesity and TNBC are highly prevalent in AA ethnicity, the expression of S100A7 was also analyzed in AA and CA TNBC patients. S100A7 is significantly highly expressed in AA TNBC women as compared to their CA counterparts (FIGS. 8A and 8B). Whole-body deletion of S100A7 drastically reduced tumor growth when implanted with aggressive murine TNBC Mvt1 cells (FIGS. 8C, 8D, and 8E).

Moreover, blood profiling and flow cytometric analyses of spleen in non-tumor bearing S100A7 floxed and KO mice revealed a significantly lower number of both peripheral blood monocytes and spleen-derived monocytes in S100A7 KO mice as compared to control floxed mice (FIGS. 8F and 8G). During breast tumorigenesis, WAT of obese individuals can be infiltrated with an increased number of circulating monocytes that will ultimately differentiate into macrophages and set up a feed-forward inflammatory process, which supports breast tumor growth and subsequent metastasis.

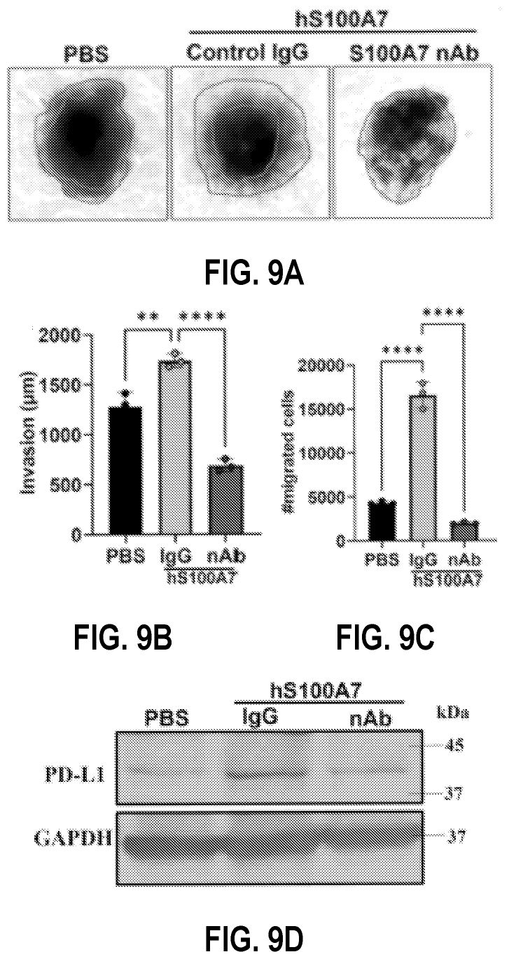

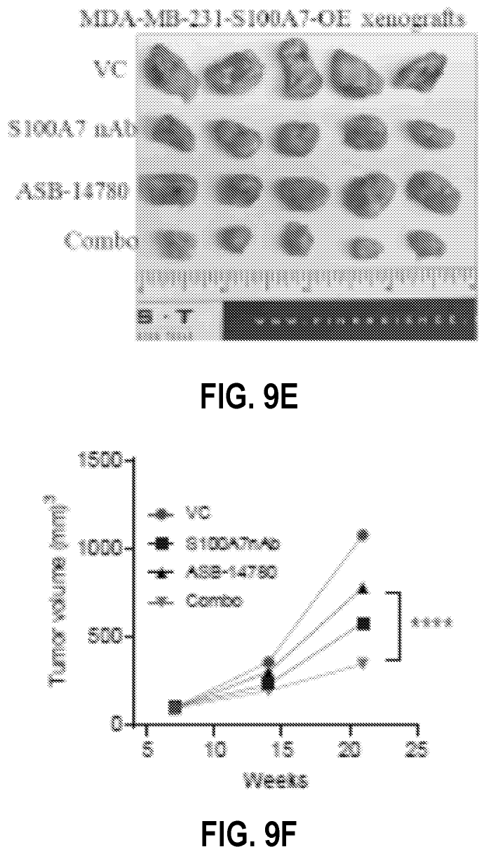

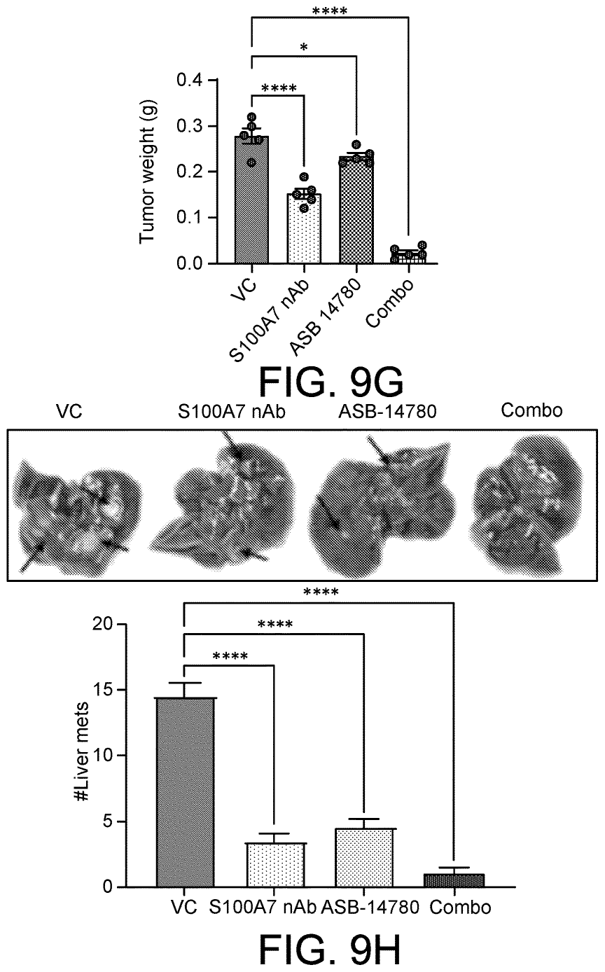

Effect of S100A7 neutralization on S100A7-induced tumorigenic effects. The translational impact and relevance of direct targeting of S100A7 in different pathophysiological conditions including breast cancer have not been determined so far. In this regard, a novel S100A7 nAb (clone 28F8-2) was generated. Here, it has been shown that S100A7 nAb inhibits the S100A7-induced tumorigenicity of TNBC cells both in-vitro and in-vivo. First, the targetability of S100A7 was evaluated in breast cancer cell migration and immunomodulation by using the generated S100A7 nAb. Its efficacy was evaluated on the invasiveness of TNBC spheroids. It was discovered that neutralization of S100A7 reduced the S100A7-mediated invasiveness of spheroid cells (FIGS. 9A and 9B). S100A7 nAb inhibits the S100A7-mediated migration of TNBC cells and decreases the expression of S100A7-induced PD-L1 expression (FIGS. 9C and 9D). Next, the effect of S100A7 nAb alone or in combination with cPLA2 inhibitor (ASB-14780) was analyzed on S10A7-induced tumor growth and metastasis. S100A7 enhances the cPLA2 expression in breast cancer cells and blockade of cPLA2 using chemical inhibitors reduced the S10A7-mediated tumor burden in vivo. S100A7 nAb in combination with the cPLA2 inhibitor significantly synergistically inhibits the S10A7-mediated TNBC growth and liver metastasis (FIGS. 9E, 9F, 9G, and 9H). In brief, S100A7 nAb is very effective in inhibiting breast tumorigenicity and is a therapeutic regimen to be used alone or in combination with other FDA-approved drugs in treating other S10A7-associated diseases including different human cancers.

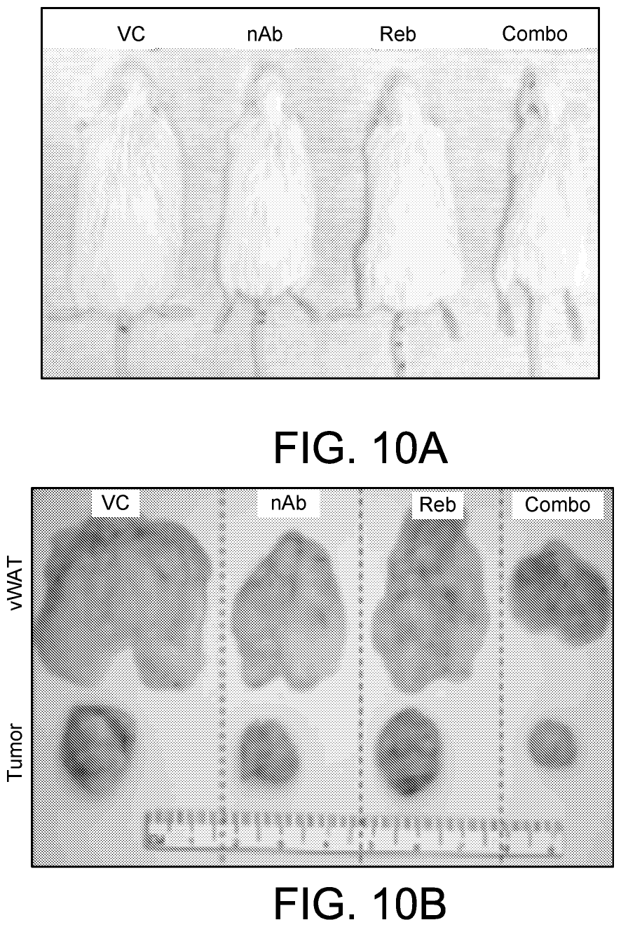

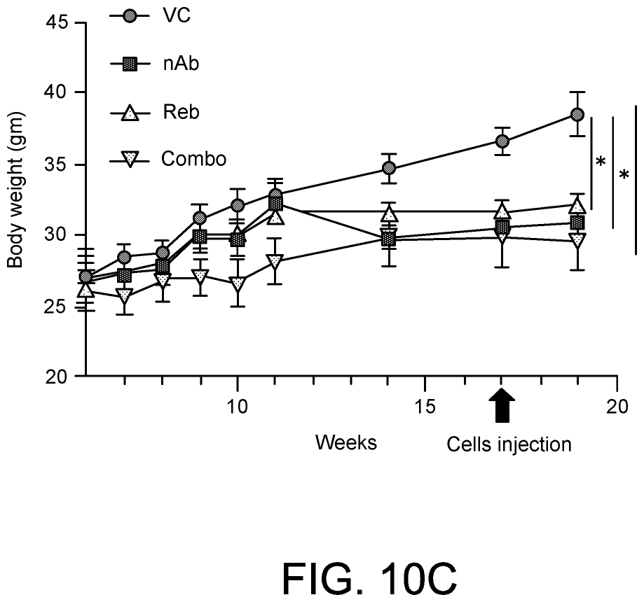

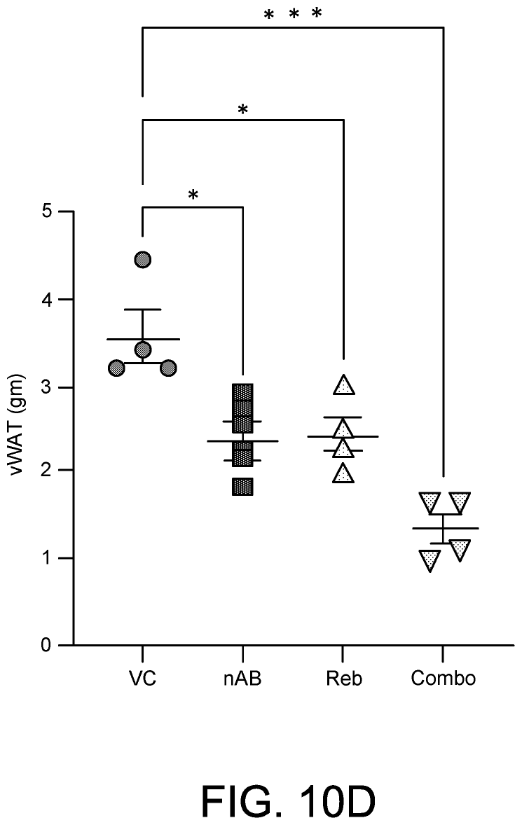

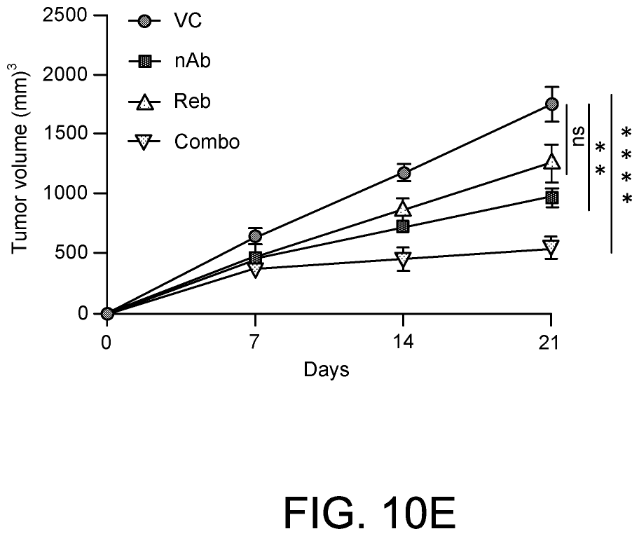

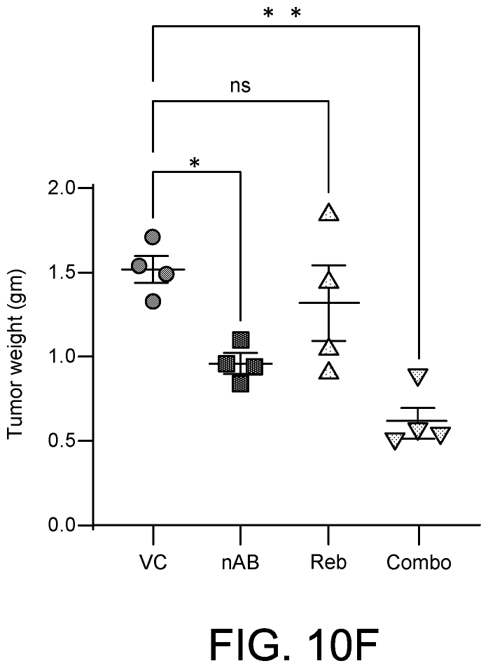

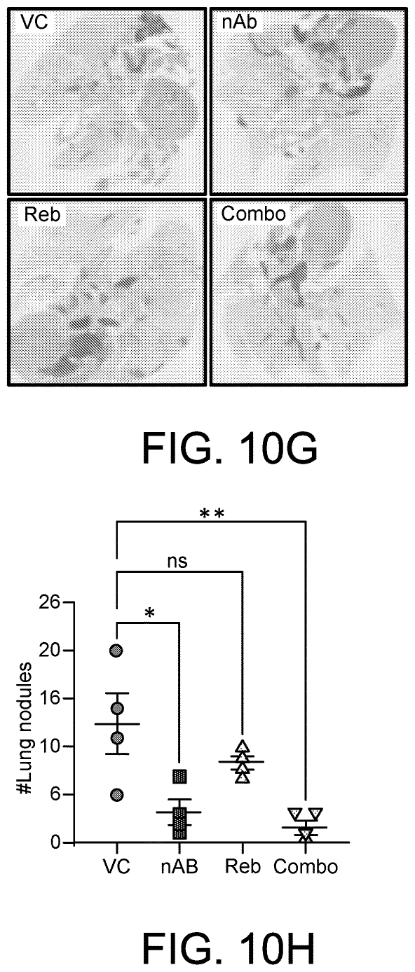

Effect of S100A7 neutralization on HFD-induced obesity and obesity-associated breast tumorigenesis. HFD-induced obesity increases the expression of S100A7 in mammary glands and vWATs, therefore the effect of S100A7 nAb on HFD-induced obesity and obesity-associated breast cancer was analyzed. For this example, the effect of S100A7 neutralization alone in combination with other drug on HFD-induced obesity and obesity-associated breast tumor growth was analyzed. First, the effect of S100A7 nAb treatment alone or in combination with SLC6A2 inhibitor (reboxetine) on HFD-induced body weight gain was determined. The genetic ablation of SLC6A2 has been recently shown to inhibit the obesity by causes browning of white fat. In addition, reboxetine has also been reported to be well tolerated and reduces depressive symptoms in breast cancer patients. Alone or combinatorial treatment of S100A7 nAb significantly inhibited the HFD-induced body weight gain and visceral fat accumulation as compared to the control IgG treated group (FIGS. 10A, 10B, 10C, and 10D). Next, the effect of S100A7 neutralization alone or in combination with reboxetine in obesity-associated breast tumor burden was analyzed using the Mvt1-generated syngeneic breast tumor model. Alone as well as combinatorial treatment of S100A7 nAb significantly reduced tumor growth and lung metastasis in these HFD-induced obese mouse models (FIGS. 10B, 10E, 10F, 10G, and 10H). Taken together, this example establishes the translational utility of the developed S100A7 nAb alone or in combination with other anti-obesity therapeutic drugs in inhibiting obesity and obesity-mediated breast tumorigenicity.

Given that over 60% of the adult population is considered overweight and 38% is considered obese in the USA alone, the wider implications of breast cancer as a co-morbidity can be devastating. This is an especially increased risk factor in women of African descent who manifest a 58.6% obesity rate. Epidemiological data have shown a strong association between obesity and breast cancer, especially TNBC initiation and progression. Moreover, obesity increases TNBC-associated mortality risk by ~30-50%. Furthermore, breast cancer is strongly associated with obesity, metabolic syndrome, and younger age of incidence. However, a molecular link between obesity and breast cancer, including TNBC has not been established. Elucidating a mechanism that links obesity with TNBC presents better therapies for these patients.

S100A7 has been shown to regulate the expression of various inflammatory molecules. S100A7 has also been shown to mediate its oncogenic effects through its receptor; RAGE. The monoclonal antibody (mAb) against RAGE suppressed the S100A7-mediated oncogenic signaling that leads to decreased lung metastasis in breast cancer. Interestingly, RAGE facilities inflammation in adipose tissue and supports the development of obesity-associated insulin resistance. Moreover, the hematopoietic insufficiency of RAGE or administration of soluble RAGE protects the mice against peripheral HFD-induced inflammation and body weight gain. However, the functional role of RAGE ligands, especially S100A7 in obesity and obesity-associated breast cancer has not been well explored.

In this example, S100A7 is shown to be highly expressed in overweight females as compared to normal-weight women. S100A7 is highly expressed in breast cancer patients. It has also been shown that S100A7 overexpression increases the growth and metastasis of TNBC cells, while its down-regulation inhibits the growth and metastasis of TNBC cells. Phylogenetic analyses have shown the mouse ancestor mS100a7a15 is most closely related to human S100A7. To elucidate the role of S100A7 in obesity-associated breast cancer, mS100a7a15 floxed (S100A7 floxed) mice and whole-body mS100a7a15 knockout (S100A7 KO) mice were generated. The whole-body deletion of S100A7 drastically inhibits breast tumor burden in KO mice injected with murine TNBC cell line. This shows that the deletion of S100A7 significantly inhibits TNBC growth. Interestingly, this also shows that the S100A7 KO mice have a protective effect against H1FD-induced obesity and showed an increased presence of multilocular lipid droplets indicative of beiging, or increased presence of brown-like adipocytes in WAT. BAT is an important tissue with regard to glucose and insulin homeostasis and also counteracts obesity. BAT is also an important tissue that can inhibit obesity and its transplantation has been shown to inhibit obesity with improved whole-body glucose metabolism. Furthermore, S100A7 whole body deletion protects the mice from HFD-induced obesity-associated glucose intolerance. In addition, S100A7 genetic inhibition suppressed the HFD-induced upregulation of different adipogenic genes such as perilipin, CD36, and PPARγ in different fat depots. Interestingly, it is also shown that deletion of S100A7 inhibits the HFD-mediated suppression of UCP1 expression in different fat depots, especially BATs. It has been reported that the absence of UCP1 enhances body weight gain and fat accumulation in mice fed an HFD. Furthermore, S100A7 deletion was found to inhibit the H1FD-induced increased secretion of leptin in blood plasma. Remarkably, the S100A7 genetic inhibition significantly reduced the H1FD-induced increased expression of leptin mRNA in vWATs. The effect of S100A7 inhibition was profiled on HFD-induced changes in the expression of mitochondrial oxidative phosphorylation complexes. Mitochondrial protein complexes are important regulators for the development of obesity. S100A7 whole body deletion differentially regulates the protein expression of all the five mitochondrial oxidative phosphorylation complexes in different depots of fat.

Tumor cells within the breast are surrounded by adipocytes, fibroblasts, and macrophages, this milieu is referred to as the tumor microenvironment (TME). The TME plays an important role in regulating the growth and metastasis of different tumors, including breast tumors. This shows that S100A7 contributes to AT expansion via increased adipogenic molecules such as PPARγ, and leptin. Whole-body deletion of S100A7 significantly reduced tumor growth in vivo. Blood profiling as well as spleen analysis also revealed that S100A7 KO mice also have a significantly lower number of monocytes as compared to wild-type mice. During breast tumorigenesis, the WAT of obese individuals can be infiltrated with an increased number of monocytes that will ultimately differentiate into macrophages and set up a feed-forward inflammatory process, which supports breast tumor growth and subsequent metastasis. Adipocytes residing in TME, especially those associated with MGs are known to attract tumor-associated macrophages (TAMs) and contribute to inflamed immunosuppressive TME, which enhances breast tumor growth and metastasis. Therefore, these results show that S100A7 inhibition is an essential therapeutic strategy to inhibit obesity, obesity-associated inflammation, and breast tumorigenesis via decreased recruitment of inflammatory myeloid cells.

To specifically target the S100A7, an S100A7 nAb was developed. Its efficacy was tested in both in-vitro and in-vivo cohorts. S100A7 nAb significantly inhibited the invasiveness of S100A7 overexpressing TNBC cells in-vitro. Therefore, the anti-tumor activity of S100A7 nAb was also analyzed alone or in combination with other anti-inflammatory drugs in vivo using S100A7 overexpressing TNBC cells. Combinatorial treatment of S100A7 nAb with cPLA2 inhibitor significantly reduced tumor growth and metastasis. S100A7 was also observed to enhance the expression of PD-L1 in TNBC cells, and treatment of S100A7 nAb reduced the S100A7-mediated overexpression of PD-L1 in these cells. Therefore, the developed S100A7 nAb can be used as an immunotherapeutic strategy to inhibit the PD-L1/PD-1-mediated signaling which drives T cells to apoptosis or into a regulatory phenotype.

Finally, the effect of S100A7 inhibition was evaluated in combination with SLC6A2 inhibitor on obesity-associated breast cancer using the S100A7 nAb. Here, it was discovered that alone or combinatorial treatment of S100A7 nAb with SLC6A2 inhibitor significantly reduced HFD-induced obesity and obesity-associated tumor burden. In summary, the role of S100A7 in obesity as well as in breast cancer was established. Furthermore, it was elucidated how to directly target a S100A7 nAb as a therapeutic strategy to treat different pathophysiological chronic inflammatory conditions associated with the abnormally high expression of S100A7.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
List of reagents and resources.

| | | |
|---|---|---|
| Reagents or resources | Source | Catalog Number |

Antibodies- Flow Cytometry

| | | |
|---|---|---|
| Anti-mouse CD45- Pacific blue | Biolegend | 157212 |
| Anti-mouse CD11b-APC; PerCP | Biolegend | 101230; 101212 |
| Anti- mouse Gr1-AF594 | Biolegend | 108448 |
| Anti- mouse Ly6C-PE | Biolegend | 128008 |

Antibody-WB and IHC

| | | |
|---|---|---|
| Anti-mouse S100A7 | Novus Biologicals | NB100-56559 |
| Anti-rabbit β-actin | Cell Signaling | 4970 |
| | Technology | |
| Anti-rabbit GAPDH | Cell Signaling | 2118 |
| | Technology | |
| Anti-rabbit α-tubulin | Cell Signaling | 2125 |
| | Technology | |
| Anti-rabbit CD36 | Invitrogen | PA1-16813 |
| Anti-rabbit FATP1 | Invitrogen | PA5-67913 |
| Anti-rabbit PPARγ | Cell Signaling | 2443 |
| | Technology | |
| Anti-rabbit Perilipin-1 | Cell Signaling | 9349 |
| | Technology | |
| Anti-rabbit UCP1 | Cell Signaling | 14670 |
| | Technology | |
| Anti-rabbit PD-L1 | Invitrogen | PA5-28115 |
| OxPhos Rodent WB Antibody | Invitrogen | 45-8099 |
| Cocktail | | |

Recombinant proteins and Reagents

| | | |
|---|---|---|
| Human recombinant S100A7 | R&D Systems | 9085-SA-050 |
| Fc receptor blocker | Biolegends | 422302 |
| Proteome Profiler Mouse | R&D Systems | ARY013 |
| Adipokine Array Kit | | |
| Fixation/ permeabilization | Thermoscientific | 00-5223-56 |
| Diluent | | |
| permeabilization buffer | Biolegends | 421002 |

Commercial assays

| | | |
|---|---|---|
| S100A7 ELISA Kit | Novus Biologicals | NBP2- |
| | | 31039/KGE004B |
| ImmPACT DAB Substrate | Vector Laboratories | SK-4100 |
| Kit, Peroxidase | | |

Abbreviation: WB, Western Blot; IHC, Immunohistochemistry

TABLE 2
Name and sequence of primers used for genotyping and qRT-PCR.

| | | | Amplicon |
|---|---|---|---|
| Genotypes | Primers pair | Sequences | size (bp) |
| Wild type | S100A7A ttRF1- | F: GAGTTCCAGGACAGCCAGAG (SEQ ID | 301 |
| | S100A7A R1 | NO. 10) | |
| | | R: GGACTCCTTCTATTCGCCATT (SEQ ID | |
| | | NO. 11) | |
| Mutant or | S100A7A ttRF1- | F: GAGTTCCAGGACAGCCAGAG (SEQ ID | 374 |
| floxed | S100A7A R1 | NO. 10) | |
| (with lox p) | | R: GGACTCCTTCTATTCGCCATT (SEQ ID | |
| | | NO. 11) | |
| Wild type | CSD-loxF- | F: GAGATGGCGCAACGCAATTAATG | No band |
| | S100A7A R1 | (SEQ ID NO. 12) | |
| | | R: GGACTCCTTCTATTCGCCATT (SEQ ID | |
| | | NO. 11) | |
| Mutant or | CSD-loxF- | F: GAGATGGCGCAACGCAATTAATG | 240 |
| floxed | S100A7A R1 | (SEQ ID NO. 12) | |
| (with lox p) | | R: GGACTCCTTCTATTCGCCATT (SEQ ID | |
| | | NO. 11) | |
| Wild type | post FLPe | F: TAGGTAGCAAAGCATGCAG (SEQ ID | 523 |
| (post FLPe) | forward-post | NO. 13) | |
| | FLPe reverse | R: TGATGTAGTATGGCTGCCT (SEQ ID | |
| | | NO. 14) | |
| Mutant or | post FLPe | F: TAGGTAGCAAAGCATGCAG (SEQ ID | 556 |
| floxed | forward-post | NO. 13) | |
| (post FLPe; | FLPe reverse | R: TGATGTAGTATGGCTGCCT (SEQ ID | |
| with lox p) | | NO. 14) | |
| Post FLPe- | SOX2-FLPe F1 | F: TCTCCCATCTCCAACAGTCC (SEQ ID | ~1500 |
| SOX2-Cre | forward- | NO. 15) | |
| (Wild) | S100A7A R1 | R: GGACTCCTTCTATTCGCCATT (SEQ ID | |
| | reverse | NO. 11) | |
| Post FLPe- | SOX2-FLPe F1 | F: TCTCCCATCTCCAACAGTCC (SEQ ID | ~526 |
| SOX2-Cre | forward- | NO. 15) | |
| (Deleted) | S100A7A R1 | R: GGACTCCTTCTATTCGCCATT (SEQ ID | |
| version 1 | reverse | NO. 11) | |
| Post FLPe- | SOX2-post FLPe | F: TAGGTAGCAAAGCATGCAG (SEQ ID | ~378 |
| SOX2-Cre | forward- | NO. 13) | |
| (Deleted) | S100A7A R1 | R: GGACTCCTTCTATTCGCCATT (SEQ ID | |
| version 2 | reverse | NO. 11) | |
| Sox-2 Cre | Forward-Reverse | F: TCATGAACTATATCCGTAACCTGGA | ~390 |
| | | (SEQ ID NO. 16) | |
| | | R: TGTTGCCAAACTCTAAACCAAATAC | |
| | | (SEQ ID NO. 17) | |
| mS100a7a | Forward-Reverse | F: GGACTCCCTCTTCCAAATCATAC (SEQ | 228 |
| 15 | primers | ID NO. 18) | |
| | | R: CCCAAGATGTACAGGAACTCATC | |
| | | (SEQ ID NO. 19) | |
| 18S rRNA | Forward-Reverse | F: CGTCGTAGTTCCGACCATAAA (SEQ ID | 254 |
| | primers | NO. 20) | |
| | | R: CGGAATCGAGAAAGAGCTATCA (SEQ | |
| | | ID NO. 21) | |

1. A recombinant antibody that specifically binds to a S100A7 polypeptide.

2. The recombinant antibody of claim 1, wherein the recombinant antibody comprises at least one heavy chain and/or at least one light chain.

3. The recombinant antibody of claim 2, wherein the heavy chain comprises at least one heavy chain complementarity determining region (CDRH) selected from CDRH1, CDRH2, and CDRH3.

4. The recombinant antibody of claim 3, wherein the CDRH1 comprises a sequence at least about 80% identical to SEQ ID NO: 1 or a fragment thereof.

5. The recombinant antibody of claim 3, wherein the CDRH2 comprises a sequence at least about 80% identical to SEQ ID NO: 2 or a fragment thereof.

6. The recombinant antibody of claim 3, wherein the CDRH3 comprises a sequence at least about 80% identical to SEQ ID NO: 3 or a fragment thereof.

7. The recombinant antibody of claim 2, wherein the heavy chain comprises a sequence at least about 80% identical to SEQ ID NO: 4 or a fragment thereof.

8. The recombinant antibody of claim 2, wherein the light chain comprises at least one light chain complementarity determining region (CDRL) selected from CDRL1, CDRL2, and CDRL3.

9. The recombinant antibody of claim 8, wherein the CDRL1 comprises a sequence at least about 80% identical to SEQ ID NO: 5 or a fragment thereof.

10. The recombinant antibody of claim 8, wherein the CDRL2 comprises a sequence at least about 80% identical to SEQ ID NO: 6 or a fragment thereof.

11. The recombinant antibody of claim 8, wherein the CDRL3 comprises a sequence at least about 80% identical to SEQ ID NO: 7 or a fragment thereof.

12. The recombinant antibody of claim 2, wherein the light chain comprises a sequence at least about 80% identical to SEQ ID NO: 8 or a fragment thereof.

13. A recombinant polynucleotide encoding the recombinant antibody of claim 1.

14. A pharmaceutical composition comprising the recombinant antibody of claim 1.

15. The pharmaceutical composition of claim 14, further comprising a cytosolic phospholipase A2 (cPLA2) inhibitor, a solute carrier family 6 member 2 (SLC6A2) inhibitor, a PD-L1 inhibitor, PD-1 inhibitor, or a combination thereof.

16.-23. (canceled)

24. A method of treating obesity or an obesity-associated disorder in a subject in need thereof comprising:
determining if the subject has obesity or an obesity-associated disorder; and
administering to the subject a therapeutically effective amount of an S100A7 inhibitor if the subject is determined to have obesity or an obesity-associated disorder.

25. The method of claim 24, wherein the subject is determined to have obesity or an obesity-associated disorder if the subject has an increased level of an S100A7 polypeptide in a biological sample obtained from the subject in comparison to a reference control.

26. The method of claim 24, wherein the S100A7 inhibitor is a polypeptide, a polynucleotide, a small molecule, or a gene editing system.

27. The method of claim 26, wherein the polynucleotide is a siRNA or a shRNA that targets an S100A7 polynucleotide.

28. The method of claim 26, wherein the gene editing system is a CRISPR/Cas endonuclease system comprising a guide RNA (gRNA) targeting an S100A7 polynucleotide.

29.-82. (canceled)
